(19) United States
(12) Patent Application Publication
(10) Pub. No.: US 2007/0244108 A1
(43) Pub. Date: Oct. 18, 2007

(54) Phenylsulfonamide Derivatives for Use as 11-Beta-Hydroxysteroid Dehydrogenase Inhibitors

(75) Inventors: Nigel Vicker, Slough (GB); Dharshini Ganeshapillai, Slough (GB); Atul Purohit, Slough (GB); Michael John Reed, Slough (GB); Barry Victor Lloyd Potter, Slough (GB)

Correspondence Address: STEPTOE & JOHNSON LLP, 1330 CONNECTICUT AVENUE, N.W., WASHINGTON, DC, 20036, US

(73) Assignee: STERIX LIMITED, BERKSHIRE (GB)

(21) Appl. No.: 11/578,999

(22) PCT Filed: Mar. 24, 2005
(86) PCT No.: PCT/GB05/01144

§ 371 Date: May 7, 2007

(30) Foreign Application Priority Data
Apr. 20, 2004 (GB) 0408771.4

(51) Int. Cl. A61K 31/535; A61K 31/445; A61P 19/08; A61P 9/00; C07D 233/00; C07D 241/04; C07D 241/00; A61P 9/12; A61P 3/10; A61K 31/4965

(52) U.S. Cl. 514/231.2; 514/255.01; 514/400; 544/164; 544/382; 548/335.1

(57) ABSTRACT

1. A compound having Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.

### FIELD OF INVENTION

The present invention relates to a compound. In particular the present invention provides compounds capable of inhibiting 11-hydroxysteroid dehydrogenase (11β-HSD).

### INTRODUCTION

The Role of Glucocorticoids

Glucocorticoids are synthesised in the adrenal cortex from cholesterol. The principle glucocorticoid in the human body is cortisol, this hormone is synthesised and secreted in response to the adrenocortictrophic hormone (ACTH) from the pituitary gland in a circadian, episodic manner, but the secretion of this hormone can also be stimulated by stress, exercise and infection. Cortisol circulates mainly bound to transcortin (cortisol binding protein) or albumin and only a small fraction is free (5-10%) for biological processes [1].

Cortisol has a wide range of physiological effects, including regulation of carbohydrate, protein and lipid metabolism, regulation of normal growth and development, influence on cognitive function, resistance to stress and mineralocorticoid activity. Cortisol works in the opposite direction compared to insulin meaning a stimulation of hepatic gluconeogenesis, inhibition of peripheral glucose uptake and increased blood glucose concentration. Glucocorticoids are also essential in the regulation of the immune response. When circulating at higher concentrations glucocorticoids are immunosuppressive and are used pharmacologically as anti-inflammatory agents.

Glucocorticoids like other steroid hormones are lipophilic and penetrate the cell membrane freely. Cortisol binds, primarily, to the intracellular glucocorticoid receptor (GR) that then acts as a transcription factor to induce the expression of glucocorticoid responsive genes, and as a result of that protein synthesis.

The Role of the 11β-HSD Enzyme

The conversion of cortisol (F) to its inactive metabolite cortisone (E) by 11β-HSD was first described in the 1950's, however it was not until later that the biological importance for this conversion was suggested [2]. In 1983 Krozowski et al. showed that the mineralocorticoid receptor (MR) has equal binding affinities for glucocorticoids and mineralocorticoids [3]. Because the circulating concentration of cortisol is a 100 times higher than that of aldosterone and during times of stress or high activity even more, it was not clear how the MR remained mineralocorticoid specific and was not constantly occupied by glucocorticoids. Earlier Ulick et al. [4] had described the hypertensive condition known as, “apparent mineralocorticoid excess” (AME), and observed that whilst secretion of aldosterone from the adrenals was in fact low the peripheral metabolism of cortisol was disrupted. These discoveries lead to the suggestion of a protective role for the enzymes. By converting cortisol to cortisone in mineralocorticoid dependent tissues 11β-HSD enzymes protects the MR from occupation by glucocorticoids and allows it to be mineralcorticoid specific. Aldosterone itself is protected from the enzyme by the presence of an aldehyde group at the C-18 position.

Congenital defects in the 11β-HSD enzyme results in over occupation of the MR by cortisol and hypertensive and hypokalemic symptoms seen in AME.

Localisation of the 11β-HSD showed that the enzyme and its activity is highly present in the MR dependent tissues, kidney and parotid. However in tissues where the MR is not mineralocorticoid specific and is normally occupied by glucocorticoids, 11 β-HSD is not present in these tissues, for example in the heart and hippocampus [5]. This research also showed that inhibition of 11 β-HSD caused a loss of the aldosterone specificity of the MR in these mineralocorticoid dependent tissues.

It has been shown that two iso-enzymes of 11 β-HSD exist. Both are members of the short chain alcohol dehydrogenase (SCAD) superfamily which have been widely conserved throughout evolution. 11 β-HSD type 2 acts as a dehydrogenase to convert the secondary alcohol group at the C-11 position of cortisol to a secondary ketone, so producing the less active metabolite cortisone. 11 β-HSD type 1 is thought to act mainly in vivo as a reductase, that is in the opposite direction to type 2 [6] [see below]. 11 βHSD type 1 and type 2 have only a 30% amino acid homology.

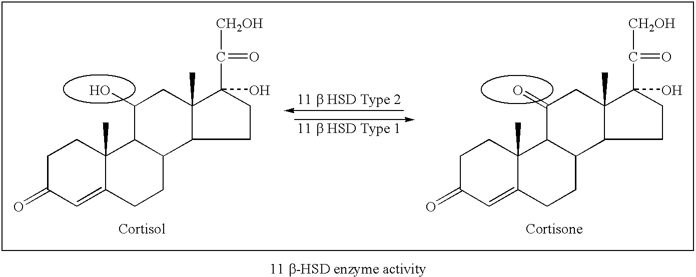

The intracellular activity of cortisol is dependent on the concentration of glucocorticoids and can be modified and independently controlled without involving the overall secretion and synthesis of the hormone.

The Role of 11 β-HSD Type 1

The direction of 11 β-HSD type 1 reaction in vivo is generally accepted to be opposite to the dehydrogenation of type 2. In vivo homozygous mice with a disrupted type 1 gene are unable to convert cortisone to cortisol, giving further evidence for the reductive activity of the enzyme [7]. 11 β-HSD type 1 is expressed in many key glucocorticoid regulated tissues like the liver, pituitary, gonad, brain, adipose and adrenals however, the function of the enzyme in many of these tissues is poorly understood [8].

The concentration of cortisone in the body is higher than that of cortisol, cortisone also binds poorly to binding globulins, making cortisone many times more biologically available. Although cortisol is secreted by the adrenal cortex, there is a growing amount of evidence that the intracellular conversion of E to F may be an important mechanism in regulating the action of glucocorticoids [9].

It may be that 11 β-HSD type 1 allows certain tissues to convert cortisone to cortisol to increase local glucocorticoid activity and potentiate adaptive response and counteracting the type 2 activity that could result in a fall in active glucocorticoids [10]. Potentiation of the stress response would be especially important in the brain and high levels of 11 β-HSD type 1 are found around the hippocampus, further proving the role of the enzyme. 11 β-HSD type 1 also seems to play an important role in hepatocyte maturation [8].

Another emerging role of the 11 β-HSD type 1 enzyme is in the detoxification process of many non-steroidal carbonyl compounds, reduction of the carbonyl group of many toxic compounds is a common way to increase solubility and therefore increase their excretion. The 11 β-HSD type 1 enzyme has recently been shown to be active in lung tissue [11]. Type 1 activity is not seen until after birth, therefore mothers who smoke during pregnancy expose their children to the harmful effects of tobacco before the child is able to metabolically detoxify this compound.

The Role of 11 β-HSD Type 2

As already stated earlier the 11 β-HSD type 2 converts cortisol to cortisone, thus protecting the MR in many key regulatory tissues of the body. The importance of protecting the MR from occupation by glucocorticoids is seen in patients with AME or liquorice intoxification. Defects or inactivity of the type 2 enzyme results in hypertensive syndromes and research has shown that patients with an hypertensive syndrome have an increased urinary excretion ratio of cortisol: cortisone. This along with a reported increase in the half life of radiolabelled cortisol suggests a reduction of 11 β-HSD type 2 activity [12].

Rationale for the Development of 11 β-HSD Inhibitors

As said earlier cortisol opposes the action of insulin meaning a stimulation of hepatic gluconeogenesis, inhibition of peripheral glucose uptake and increased blood glucose concentration. The effects of cortisol appear to be enhanced in patients suffering from glucose intolerance or diabetes mellitus. Inhibition of the enzyme 11 β-HSD type 1 would increase glucose uptake and inhibit hepatic gluconeogenesis, giving a reduction in circulatory glucose levels. The development of a potent 11 β-HSD type 1 inhibitor could therefore have considerable therapeutic potential for conditions associated with elevated blood glucose levels.

An excess in glucocorticoids can result in neuronal dysfunctions and also impair cognitive functions. A specific 11 β-HSD type 1 inhibitor might be of some importance by reducing neuronal dysfunctions and the loss of cognitive functions associated with ageing, by blocking the conversion of cortisone to cortisol.

Glucocorticoids also have an important role in regulating part of the immune response [13]. Glucocorticoids can suppress the production of cytokines and regulate the receptor levels. They are also involved in determining whether T-helper (Th) lymphocytes progress into either Th1 or Th2 phenotype. These two different types of Th cells secrete a different profile of cytokines, Th2 is predominant in a glucocorticoid environment. By inhibiting 11 β-HSD type 1, Th1 cytokine response would be favoured. It is also possible to inhibit 11 β-HSD type 2, thus by inhibiting the inactivation of cortisol, it may be possible to potentiate the anti-inflammatory effects of glucocorticoids.

Aspects of the invention are defined in the appended claims.

### SUMMARY ASPECTS OF THE PRESENT INVENTION

In one aspect the present invention provides a compound having Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.

In one aspect the present invention provides a pharmaceutical composition comprising

(i) a compound having Formula
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present;
(ii) optionally admixed with a pharmaceutically acceptable carrier, diluent, excipient or adjuvant.

In one aspect the present invention provides a compound for use in medicine wherein the compound has Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.

In one aspect the present invention provides a use of a compound in the manufacture of a medicament for use in the therapy of a condition or disease associated with 11β-HSD, wherein the compound has Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.
Some Advantages

One key advantage of the present invention is that the compounds of the present invention can act as 11 β-HSD inhibitors. The compounds may inhibit the interconversion of inactive 11-keto steroids with their active hydroxy equivalents. Thus present invention provides methods by which the conversion of the inactive to the active form may be controlled, and to useful therapeutic effects which may be obtained as a result of such control. More specifically, but not exclusively, the invention is concerned with interconversion between cortisone and cortisol in humans.

Another advantage of the compounds of the present invention is that they may be potent 11β-HSD inhibitors in vivo.

Some of the compounds of the present invention are also advantageous in that they may be orally active.

The present invention may provide for a medicament for one or more of (i) regulation of carbohydrate metabolism, (ii) regulation of protein metabolism, (iii) regulation of lipid metabolism, (iv) regulation of normal growth and/or development, (v) influence on cognitive function, (vi) resistance to stress and mineralocorticoid activity.

Some of the compounds of the present invention may also be useful for inhibiting hepatic gluconeogenesis. The present invention may also provide a medicament to relieve the effects of endogenous glucocorticoids in diabetes mellitus, obesity (including centripetal obesity), neuronal loss and/or the cognitive impairment of old age. Thus, in a further aspect, the invention provides the use of an inhibitor of 11β-HSD in the manufacture of a medicament for producing one or more therapeutic effects in a patient to whom the medicament is administered, said therapeutic effects selected from inhibition of hepatic gluconeogenesis, an increase in insulin sensitivity in adipose tissue and muscle, and the prevention of or reduction in neuronal loss/cognitive impairment due to glucocorticoid-potentiated neurotoxicity or neural dysfunction or damage.

From an alternative point of view, the invention provides a method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, and any combination of the aforementioned conditions, the method comprising the step of administering to said patient a medicament comprising a pharmaceutically active amount of a compound in accordance with the present invention.

Some of the compounds of the present invention may be useful for the treatment of cancer, such as breast cancer, as well as (or in the alternative) non-malignant conditions, such as the prevention of auto-immune diseases, particularly when pharmaceuticals may need to be administered from an early age.

### DETAILED ASPECTS OF THE PRESENT INVENTION

As previously mentioned, in one aspect the present invention provides a compound having Formula I defined above.

As previously mentioned, in one aspect the present invention provides a pharmaceutical composition comprising

(i) a compound having Formula I defined above

(ii) optionally admixed with a pharmaceutically acceptable carrier, diluent, excipient or adjuvant.

As previously mentioned, in one aspect the present invention provides a compound having Formula I defined above, for use in medicine.

As previously mentioned, in one aspect the present invention provides a use of a compound having Formula I defined above in the manufacture of a medicament for use in the therapy of a condition or disease associated with 11β-HSD.

In one aspect the present invention provides a use of a compound having Formula I defined above in the manufacture of a medicament for use in the therapy of a condition or disease associated with adverse 11β-HSD levels.

In one aspect the present invention provides a use of a compound having Formula I defined above in the manufacture of a pharmaceutical for modulating 11β-HSD activity.

In one aspect the present invention provides a use of a compound having Formula I defined above in the manufacture of a pharmaceutical for inhibiting 11β-HSD activity.

In one aspect the present invention provides a method comprising (a) performing a 11β-HSD assay with one or more candidate compounds having Formula I defined above; (b) determining whether one or more of said candidate compounds is/are capable of modulating 11β-HSD activity; and (c) selecting one or more of said candidate compounds that is/are capable of modulating 11β-HSD activity.

In one aspect the present invention provides a method comprising (a) performing a 11β-HSD assay with one or more candidate compounds having Formula I defined above; (b) determining whether one or more of said candidate compounds is/are capable of inhibiting 11β-HSD activity; and (c) selecting one or more of said candidate compounds that is/are capable of inhibiting 11β-HSD activity.

In one aspect the present invention provides
a compound identified by the above method,
the use of the said compound in medicine,
a pharmaceutical composition comprising the said compound, optionally admixed with a pharmaceutically acceptable carrier, diluent, excipient or adjuvant,
use of the said compound in the manufacture of a medicament for use in the therapy of a condition or disease associated with 11β-HSD, and
use of the said compound in the manufacture of a medicament for use in the therapy of a condition or disease associated with adverse 11β-HSD levels.

For ease of reference, these and further aspects of the present invention are now discussed under appropriate section headings. However, the teachings under each section are not necessarily limited to each particular section.

### Preferable Aspects

Compound

As previously mentioned, in one aspect the present invention provides a compound having Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group. R₁, R₂, R₃ and R₄

R₁ is an optionally substituted phenyl ring and R₂ is a heterocyclic ring. R₁ and R₂ are referred to collectively as the ring systems.

R₁ is an optionally substituted phenyl ring.

R₁ may be substituted or unsubstituted. Preferably R₁ is substituted.

R₁ may be substituted with one or more hydrocarbon groups.

Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

More preferably R₁ is substituted with one or more alkyl groups such as one or more C₁₋₅ alkyl groups. More preferably the substituents are selected from methyl, ethyl and propyl, preferably propyl.

In one preferred embodiment, R₁ is substituted at the para position of the phenyl ring. Preferably, R₁ is substituted with a C₁₋₅ alkyl group at the para position of the phenyl ring.

Preferably R₁ is 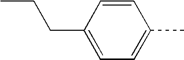
R₂ is a heterocyclic ring. R₂ may be aromatic or non-aromatic. R₂ may be substituted or unsubstituted. Preferably R₂ is a five or six membered ring.

In one preferred aspect, R₂ is a six membered non-aromatic heterocyclic ring.

In another preferred aspect R₂ is five membered aromatic heterocyclic ring.

Preferably R₂ comprises carbon and a hetero atom selected from O and N.

In one preferred aspect, R₂ has the formula 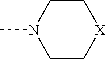
wherein X is selected from the group consisting of S, O and NR₄ wherein R₄ is H or a hydrocarbyl group. Preferably X is selected from the group consisting of O and NR₄.

In one preferred aspect, R₂ comprises carbon and two hetero atoms selected from O and N.

In a highly preferred aspect, the atoms in the heterocyclic ring of R₂ are selected from C, O and N. More preferably the atoms in the heterocyclic ring of R₂ are C atoms and exactly two heterocyclic atoms selected from O and N. It will be readily appreciated that in this context the “atoms in the heterocyclic ring” refers to those atoms which are covalently bonded to each other in a closed loop, however R₂ may also contain other atoms such as H atoms or atoms of groups substituted on the heterocyclic ring.

In one aspect, the heterocyclic ring of R₂ does not contain any S atoms. In one aspect R₂ does not contain any S atoms.

In a highly preferred aspect, R₂ is selected from the group consisting of:

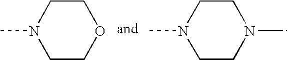

In another preferred aspect, R₂ is

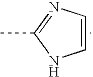

R₂ may be substituted or unsubstituted. In one aspect, R₂ is substituted. In this aspect, preferably R₂ is substituted with one or more hydrocarbon groups. More preferably R₂ is substituted with one or more alkyl groups such as one or more C₁₋₅ alkyl groups. Preferably R₂ is substituted with one or more groups selected from methyl, ethyl and propyl.

The compound of the present invention may have substituents other than those of the ring systems show herein. Furthermore the ring systems herein are given as general formulae and should be interpreted as such. The absence of any specifically shown substituents on a given ring member indicates that the ring member may substituted with any moiety of which H is only one example. Each ring system may contain one or more degrees of unsaturation, for example is some aspects one or more rings of a ring system is aromatic. Each ring system may be carbocyclic or may contain one or more hetero atoms.

The compound of the invention, in particular the ring systems of the compound of the invention may contain substituents other than those show herein. By way of example, these other substituents may be one or more of: one or more halo groups, one or more O groups, one or more hydroxy groups, one or more amino groups, one or more sulphur containing group(s), one or more hydrocarbyl group(s)—such as an oxyhydrocarbyl group.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In some aspects of the present invention, one or more hydrocarbyl groups is independently selected from optionally substituted alkyl group, optionally substituted haloalkyl group, aryl group, alkylaryl group, alkylarylakyl group, and an alkene group.

In some aspects of the present invention, one or more hydrocarbyl groups is independently selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

In some aspects of the present invention, one or more hydrocarbyl groups is independently selected from C₁-C₁₀ haloalkyl group, C₁-C₆ haloalkyl group, C₁-C₃ haloalkyl group, C₁-C₁₀ bromoalkyl group, C₁-C₆ bromoalkyl group, and C₁-C₃ bromoalkyl group. Typical haloalkyl groups include C₁ haloalkyl, C₂ haloalkyl, C₃ haloalkyl, C₄ haloalkyl, C₅ haloalkyl, C₇ haloalkyl, C₈ haloalkyl, C₁ bromoalkyl, C₂ bromoalkyl, C₃ bromoalkyl, C₄ bromoalkyl, C₅ bromoalkyl, C₇ bromoalkyl, and C₈ bromoalkyl.

In some aspects of the present invention, one or more hydrocarbyl groups is independently selected from aryl groups, alkylaryl groups, alkylarylakyl groups, —(CH₂)₁₋₁₀-aryl, —(CH₂)₁₋₁₀-Ph, (CH₂)₁₋₁₀-Ph-C₁₋₁₀ alkyl, —(CH₂)₁₋₅-Ph, (CH₂)₁₋₅-Ph-C₁₋₅ alkyl, —(CH₂)₁₋₃-Ph, (CH₂)₁₋₃-Ph-C₁₋₃ alkyl, —CH₂-Ph, and —CH₂-Ph-C(CH₃)₃. The aryl groups may contain a hetero atom. Thus the aryl group or one or more of the aryl groups may be carbocyclic or more may heterocyclic. Typical hetero atoms include O, N and S, in particular N.

In some aspects of the present invention, one or more hydrocarbyl groups is independently selected from —(CH₂)₁₋₁₀-cycloalkyl, —(CH₂)₁₋₁₀—C₃₋₁₀cycloalkyl, —(CH₂)₁₋₇—C₃₋₇cycloalkyl, —(CH₂)₁₋₅—C₃₋₅cycloalkyl, —(CH₂)₁₋₃—C₃₋₅cycloalkyl, and —CH₂—C₃cycloalkyl.

In some aspects of the present invention, one or more hydrocarbyl groups is independently selected from alkene groups. Typical alkene groups include C₁-C₁₀ alkene group, C₁-C₆ alkene group, C₁-C₃ alkene group, such as C₁, C₂, C₃, C₄, C₅, C₆, or C₇ alkene group. In a preferred aspect the alkene group contains 1, 2 or 3 C═C bonds. In a preferred aspect the alkene group contains 1 C═C bond. In some preferred aspect at least one C═C bond or the only C═C bond is to the terminal C of the alkene chain, that is the bond is at the distal end of the chain to the ring system.

In some aspects of the present invention, one or more hydrocarbyl groups is independently selected from oxyhydrocarbyl groups.

The term “oxyhydrocarbyl” group as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Typically, the oxyhydrocarbyl group is of the formula C₁₋₆O (such as a C₁₋₃O).

In general terms the ring systems of the present compounds may contain a variety of non-interfering substituents. In particular, the ring systems may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

As previously mentioned, in one aspect, the present invention provides a compound having Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker; wherein when R₂ is a five-membered aromatic heterocyclic ring, L is present.

Preferably R₃ is H or a hydrocarbon group. More preferably R₃ is H or an alkyl group. More preferably R₃ is H or a C₁₋₅ alkyl group, for example R₃ is H or methyl, ethyl, propyl, butyl or pentyl, preferably H or methyl. In a highly preferred embodiment R₃ is H.

As previously mentioned, in one preferred aspect, R₂ has the formula 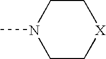
wherein X is selected from the group consisting of S, O and NR₄ wherein R₄ is H or a hydrocarbyl group.

Preferably R₄ is H or a hydrocarbon group. More preferably R₄ is H or an alkyl group. More preferably R₄ is H or a C₁₋₅ alkyl group, for example R₄ is H or methyl, ethyl, propyl, butyl or pentyl, preferably H or methyl. In one aspect preferably R₄ is H. In a highly preferred embodiment R₄ is methyl.

L

As previously mentioned, the present invention provides a compound having Formula I
R₁—SO₂NR₃-L-R₂ Formula I
wherein R₁ is an optionally substituted phenyl ring; R₂ is a heterocyclic ring; R₃ is H or a hydrocarbyl group; and L is an optional acyclic linker wherein when R₂ is a five-membered aromatic heterocyclic ring, L is present.

In one aspect, preferably L is present.

Preferably, L is a divalent hydrocarbyl linker group. More preferably, L has the formula C_{y}H_{2y} wherein y is an integer from 1 to 10. Preferably y is an integer from 1 to 5, more preferably 1 to 3 such as 1, 2 or 3.

In another preferred aspect, L has the formula (CH₂)ₙ wherein n is an integer from 1 to 10. Preferably n is an integer from 1 to 5, more preferably 1 to 3 such as 1, 2 or 3. In a highly preferred aspect, L is —CH₂CH₂—.

Further Aspects

For some applications, preferably the compounds have a reversible action.

For some applications, preferably the compounds have an irreversible action.

In one embodiment, the compounds of the present invention are useful for the treatment of breast cancer.

The compounds of the present invention may be in the form of a salt.

The present invention also covers novel intermediates that are useful to prepare the compounds of the present invention. For example, the present invention covers novel alcohol precursors for the compounds. The present invention also encompasses a process comprising precursors for the synthesis of the compounds of the present invention.

Steroid Dehydrogenase

11βSteroid dehydrogenase may be referred to as “11β-HSD” or “HD” for short

In some aspects of the invention 11β-HSD is preferably 11β-HSD Type 1.

In some aspects of the invention 11β-HSD is preferably 11β-HSD Type 2.

Steroid Dehydrogenase Inhibition

It is believed that some disease conditions associated with HD activity are due to conversion of a inactive, cortisone to an active, cortisol. In disease conditions associated with HD activity, it would be desirable to inhibit HD activity.

Here, the term “inhibit” includes reduce and/or eliminate and/or mask and/or prevent the detrimental action of HD.

HD Inhibitor

In accordance with the present invention, the compound of the present invention is capable of acting as an HD inhibitor.

Here, the term “inhibitor” as used herein with respect to the compound of the present invention means a compound that can inhibit HD activity—such as reduce and/or eliminate and/or mask and/or prevent the detrimental action of HD. The HD inhibitor may act as an antagonist.

The ability of compounds to inhibit steroid dehydrogenase activity can be assessed using the suitable biological assay presented in the Examples section.

It is to be noted that the compound of the present invention may have other beneficial properties in addition to or in the alternative to its ability to inhibit HD activity.

Therapy

The compounds of the present invention may be used as therapeutic agents—i.e. in therapy applications.

The term “therapy” includes curative effects, alleviation effects, and prophylactic effects.

The therapy may be on humans or animals, preferably female animals.

Pharmaceutical Compositions

In one aspect, the present invention provides a pharmaceutical composition, which comprises a compound according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Combination Pharmaceutical

The compound of the present invention may be used in combination with one or more other active agents, such as one or more other pharmaceutically active agents.

By way of example, the compounds of the present invention may be used in combination with other 11 β-HSD inhibitors and/or other inhibitors such as an aromatase inhibitor (such as for example, 4hydroxyandrostenedione (4-OHA)), and/or a steroid sulphatase inhibitors such as EMATE and/or steroids—such as the naturally occurring sterneurosteroids dehydroepiandrosterone sulfate (DHEAS) and pregnenolone sulfate (PS) and/or other structurally similar organic compounds.

In addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the compounds of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

Cell Cycling

The compounds of the present invention may be useful in the method of treatment of a cell cycling disorder.

As discussed in “Molecular Cell Biology” 3rd Ed. Lodish et al. pages 177-181 different eukaryotic cells can grow and divide at quite different rates. Yeast cells, for example, can divide every 120 min., and the first divisions of fertilised eggs in the embryonic cells of sea urchins and insects take only 1530 min. because one large pre-existing cell is subdivided. However, most growing plant and animal cells take 10-20 hours to double in number, and some duplicate at a much slower rate. Many cells in adults, such as nerve cells and striated muscle cells, do not divide at all; others, like the fibroblasts that assist in healing wounds, grow on demand but are otherwise quiescent.

Still, every eukaryotic cell that divides must be ready to donate equal genetic material to two daughter cells. DNA synthesis in eukaryotes does not occur throughout the cell division cycle but is restricted to a part of it before cell division.

The relationship between eukaryotic DNA synthesis and cell division has been thoroughly analysed in cultures of mammalian cells that were all capable of growth and division. In contrast to bacteria, it was found, eukaryotic cells spend only a part of their time in DNA synthesis, and it is completed hours before cell division (mitosis). Thus a gap of time occurs after DNA synthesis and before cell division; another gap was found to occur after division and before the next round of DNA synthesis. This analysis led to the conclusion that the eukaryotic cell cycle consists of an M (mitotic) phase, a G₁ phase (the first gap), the S (DNA synthesis) phase, a G₂ phase (the second gap), and back to M. The phases between mitoses (G₁, S, and G₂) are known collectively as the interphase.

Many nondividing cells in tissues (for example, all quiescent fibroblasts) suspend the cycle after mitosis and just prior to DNA synthesis; such “resting” cells are said to have exited from the cell cycle and to be in the G₀ state.

It is possible to identify cells when they are in one of the three interphase stages of the cell cycle, by using a fluorescence-activated cell sorter (FACS) to measure their relative DNA content: a cell that is in G₁ (before DNA synthesis) has a defined amount x of DNA; during S (DNA replication), it has between x and 2x; and when in G₂ (or M), it has 2x of DNA.

The stages of mitosis and cytokinesis in an animal cell are as follows

(a) Interphase. The G₂ stage of interphase immediately precedes the beginning of mitosis. Chromosomal DNA has been replicated and bound to protein during the S phase, but chromosomes are not yet seen as distinct structures. The nucleolus is the only nuclear substructure that is visible under light microscope. In a diploid cell before DNA replication there are two morphologic chromosomes of each type, and the cell is said to be 2n. In G₂, after DNA replication, the cell is 4n. There are four copies of each chromosomal DNA. Since the sister chromosomes have not yet separated from each other, they are called sister chromatids.

b) Early prophase. Centrioles, each with a newly formed daughter centriole, begin moving toward opposite poles of the cell; the chromosomes can be seen as long threads. The nuclear membrane begins to disaggregate into small vesicles.

(c) Middle and late prophase. Chromosome condensation is completed; each visible chromosome structure is composed of two chromatids held together at their centromeres. Each chromatid contains one of the two newly replicated daughter DNA molecules. The microtubular spindle begins to radiate from the regions just adjacent to the centrioles, which are moving closer to their poles. Some spindle fibres reach from pole to pole; most go to chromatids and attach at kinetochores.

(d) Metaphase. The chromosomes move toward the equator of the cell, where they become aligned in the equatorial plane. The sister chromatids have not yet separated.

(e) Anaphase. The two sister chromatids separate into independent chromosomes. Each contains a centromere that is linked by a spindle fibre to one pole, to which it moves. Thus one copy of each chromosome is donated to each daughter cell. Simultaneously, the cell elongates, as do the pole-to-pole spindles. Cytokinesis begins as the cleavage furrow starts to form.

(f) Telophase. New membranes form around the daughter nuclei; the chromosomes uncoil and become less distinct, the nucleolus becomes visible again, and the nuclear membrane forms around each daughter nucleus. Cytokinesis is nearly complete, and the spindle disappears as the microtubules and other fibres depolymerise. Throughout mitosis the “daughter” centriole at each pole grows until it is full-length. At telophase the duplication of each of the original centrioles is completed, and new daughter centrioles will be generated during the next interphase.

(g) Interphase. Upon the completion of cytokinesis, the cell enters the G₁ phase of the cell cycle and proceeds again around the cycle.

It will be appreciated that cell cycling is an extremely important cell process. Deviations from normal cell cycling can result in a number of medical disorders. Increased and/or unrestricted cell cycling may result in cancer. Reduced cell cycling may result in degenerative conditions. Use of the compound of the present invention may provide a means to treat such disorders and conditions.

Thus, the compound of the present invention may be suitable for use in the treatment of cell cycling disorders such as cancers, including hormone dependent and hormone independent cancers.

In addition, the compound of the present invention may be suitable for the treatment of cancers such as breast cancer, ovarian cancer, endometrial cancer, sarcomas, melanomas, prostate cancer, pancreatic cancer etc. and other solid tumours. For some applications, cell cycling is inhibited and/or prevented and/or arrested, preferably wherein cell cycling is prevented and/or arrested. In one aspect cell cycling may be inhibited and/or prevented and/or arrested in the G₂/M phase. In one aspect cell cycling may be irreversibly prevented and/or inhibited and/or arrested, preferably wherein cell cycling is irreversibly prevented and/or arrested.

By the term “irreversibly prevented and/or inhibited and/or arrested” it is meant after application of a compound of the present invention, on removal of the compound the effects of the compound, namely prevention and/or inhibition and/or arrest of cell cycling, are still observable. More particularly by the term “irreversibly prevented and/or inhibited and/or arrested” it is meant that when assayed in accordance with the cell cycling assay protocol presented herein, cells treated with a compound of interest show less growth after Stage 2 of the protocol I than control cells. Details on this protocol are presented below.

Thus, the present invention provides compounds which: cause inhibition of growth of oestrogen receptor positive (ER+) and ER negative (ER−) breast cancer cells in vitro by preventing and/or inhibiting and/or arresting cell cycling; and/or cause regression of nitroso-methyl urea (NMU)-induced mammary tumours in intact animals (i.e. not ovariectomised), and/or prevent and/or inhibit and/or arrest cell cycling in cancer cells; and/or act in vivo by preventing and/or inhibiting and/or arresting cell cycling and/or act as a cell cycling agonist.

### Cell Cycling Assay

### Protocol 1

Procedure

Stage 1

MCF-7 breast cancer cells are seeded into multi-well culture plates at a density of 105 cells/well. Cells were allowed to attach and grown until about 30% confluent when they are treated as follows:

Control—no treatment

Compound of Interest (COI) 20 μM

Cells are grown for 6 days in growth medium containing the COI with changes of medium/COI every 3 days. At the end of this period cell numbers were counted using a Coulter cell counter.

Stage 2

After treatment of cells for a 6-day period with the COI cells are re-seeded at a density of 10⁴ cells/well. No further treatments are added. Cells are allowed to continue to grow for a further 6 days in the presence of growth medium. At the end of this period cell numbers are again counted.

Cancer

As indicated, the compounds of the present invention may be useful in the treatment of a cell cycling disorder. A particular cell cycling disorder is cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

Other Therapies

As previously mentioned, in one aspect the present invention provides use of a compound as described herein in the manufacture of a medicament for use in the therapy of a condition or disease associated with 11β-HSD.

Conditions and diseases associated with 11β-HSD have been reviewed in Walker, E. A,; Stewart, P. M.; Trends in Endocrinology and Metabolism, 2003, 14 (7), 334-339.

In a preferred aspect, the condition or disease is selected from the group consisting of:
metabolic disorders, such as diabetes nd obesity
cardiovascular disorders, such as hypertension
glaucoma
inflammatory disorders, such as arthritis or asthma
immune disorders
bone disorders, such as osteoporosis
cancer
intra-uterine growth retardation
apparent mineralocorticoid excess syndrome (AME)
polycystic ovary syndrome (PCOS)
hirsutism
acne
oligo- or amenorrhea
adrenal cortical adenoma and carcinoma
Cushing's syndrome
pituitary tumours
invasive carcinomas
breast cancer; and
endometrial cancer.

It is also to be understood that the compound/composition of the present invention may have other important medical implications.

For example, the compound or composition of the present invention may be useful in the treatment of the disorders listed in WO-A-99/52890—viz:

In addition, or in the alternative, the compound or composition of the present invention may be useful in the treatment of the disorders listed in WO-A-98/05635. For ease of reference, part of that list is now provided: diabetes including Type II diabetes, obesity, cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### SUMMARY

In summation, the present invention provides compounds for use as steroid dehydrogenase inhibitors, and pharmaceutical compositions for the same.

### BRIEF DESCRIPTION OF THE FIGURES

The present invention will be described in further detail by way of example only with reference to the accompanying figures in which:

FIG. 1 is a graph showing extraction efficiencies obtained with four extraction methods.

FIG. 2 is a graph showing a comparison of 11β-HSD1 activity in rat and human hepatic microsomes.

FIG. 3 is a series of graphs showing the effect of incubation time on human microsomal 11β-HSD1 activity

FIG. 4 is a series of graphs showing the effect of microsomal protein concentration on human microsomal 11β-HSD1 activity.

FIG. 5 is a graph showing the substrate (cortisone) saturation curve for human hepatic microsomal 11β HSD1.

FIG. 6 is a Lineweaver-Burke plot.

FIG. 7 is a graph showing the IC₅₀ determination for Glycyrrhetinic acid.

FIG. 8 is a graph showing the IC₅₀ determination for Carbenoxolone.

FIGS. 9(A), 9(B) and 9(C) are graphs showing the 11 β-HSD1 activity measured by Immunoassay. FIG. 9(A) shows the effect of protein; FIG. 9(B) shows the effect of cortisone; and FIG. 9(C) shows the effect of Tween-80.

FIG. 10 is a graph showing the performance of the cortisol immunoassay: various experimental designs.

FIG. 11 is a graph showing the effect of increasing microsomal protein on measurement of 11β HSD1 activity detected by Assay Designs Immunoassay.

FIG. 12 is a graph showing the detection of 11γ HSD1 activity by RIA using the Immunotech anti-cortisol antibody.

FIG. 13 is a graph showing the effect of lowering the Immunotech antibody concentration on the signal to noise (microsome group compared to GA blank group).

FIG. 14 is a graph showing the Immunotech antibody saturation curve for detection of 11β HSD1 activity by RIA.

FIG. 15 is a graph showing the linearity of human hepatic microsomal 11β HSD1 activity detected by RIA.

FIG. 16 is a graph showing the effect of Tween 80 on detection of human hepatic microsomal 11β HSD1 activity by RIA.

FIG. 17 is a graph showing the effect of buffer systems on detection of human hepatic microsomal 11β HDS1 activity by RIA.

FIG. 18 is a graph showing the linearity of human hepatic microsomal 11β HSD1 activity with incubation time detected by RIA.

FIG. 19 is a graph showing the substrate saturation curve for human hepatic microsomal 11β HDS1 activity detected by RIA.

FIG. 20 is a Lineweaver-Burke plot

FIG. 21 is an IC₅₀ curve for inhibition of human hepatic microsomal 11β HSD1 activity by Glycyrrhetinic acid.

FIG. 22 is an IC₅₀ curve for inhibition of human hepatic microsomal 11β HSD1 activity by Glycyrrhetinic acid in the presence of 350 nM cortisone.

FIG. 23 is an IC₅₀ curve for inhibition of human hepatic microsomal 11β HSD1 activity by Carbenoxolone in the presence of 350 nM cortisone.

The present invention will now be described in further detail in the following examples.

### EXAMPLES

The present invention will now be described only by way of example.

Biological Assays

Standard Operating Procedure for the 11β-Hydroxysteroid Dehydrogenase Type 1 cortisol Radioimmunoassay (11β HSD1 Cortisol RIA).

Reagents

Cortisone, Cortisol (Hydrocortisone), NADPH, Glucose-6-phosphate, Glycyrrhetinic acid (GA), Dextran coated charcoal (C6197) and DMSO were obtained from Sigma Aldrich, Carbenoxolone was obtained from ICN Biomedicals, Product 215493001, ³H-cortisone was obtained from American Radiolabelled Compounds Inc, Product ART-743, ³H-cortisol was obtained from NEN, Product NET 396, ¹⁴C-cortisol was obtained from NEN, Product NEC 163, human hepatic microsomes were obtained from XenoTech, product H0610/Lot 0210078, rat hepatic microsomes were obtained from XenoTech, SPA beads were obtained from Amersham, Product RPNQ0017, the Immunoassay kit was obtained from Assay Designs, Product 900-071, the Immunologicals Direct anti-cortisol antibody was Product OBT 0646, the Sigma anti-cortisol antibody was Product C8409 and the Immunotech antibody was supplied by Beckman, Product IMBULK3 6D6.

Buffer Solutions

Buffer 1, from Barf et al., (2002) [14]: 30 mM Tris-HCL, pH 7.2, containing 1 mM EDTA

Buffer 2, from the Sterix protocol: PBS (pH 7.4) containing 0.25M sucrose

Buffer 3, from the Sigma RIA protocol: 50 mM Tris-HCL, pH 8, containing 0.1 M NaCl and 0.1% gelatin

Stop solution, from Barf et al., (2002) [14]: 1 mM Glycyrrhetinic acid in 100% DMSO

Enzyme assays were carried out in the presence of 181 μM NADPH, 1 mM Glucose-6-Phosphate and cortisone concentrations indicated for each experiment.

Enzyme Assay Buffer

30 mM Tris-HCL, pH 7.2 containing 1 mM EDTA

Antibody Binding Buffer

50 mM Tris-HCL, pH 8, containing 0.1 M NaCl and 0.1% gelatin

Compound Preparation

Prepare 10 mM stock solutions in 100% DMSO at 100 times the required assay concentration. Dilute into assay buffer 1 in 25. Also dilute neat DMSO 1 in 25 into assay buffer for controls.

Substrate Preparation

Prepare a solution of cortisone in ethanol 600 times the required assay concentration (175 nM). Dilute this 1 in 50 into assay buffer.

Prepare NADPH as a 1.8 mg/ml solution in assay buffer.

Prepare G-6-P as a 3.65 mg/ml solution in assay buffer.

Mix these 3 solutions 1:1:1 to make a solution of sufficient volume for 25 μl additions to each sample. Add 0.5 μCi tritiated cortisone per 25 μl and mix the solution well.

Microsome Preparation

Dilute stock 20 mg/ml solution 1 in 100 with assay buffer.

Antibody Preparation

Dilute stock antibody solution to 17 μg/ml in antibody binding buffer.

Dextran Coated Charcoal Preparation

Make a 20 mg/ml solution in antibody binding buffer and chill on ice.

Enzyme Assay

To a u-bottom polypropylene 96 well plate add:

25 μl compound dilution or diluted DMSO to controls, NSB's and blanks

10 μl mM GA in DMSO (enzyme stop solution) to blanks

25 μl substrate mixture to all samples

50 μl diluted microsomes to all samples

Incubate plate for 30 min at 37° C. shaking

Add 10 μl enzyme stop solution to all wells except the blanks

Add 100 μl antibody solution to all wells except the NSB's, add antibody binding buffer to these wells

Incubate at 37° C. for 1 h

Chill plate on ice for 15 min

Add 50 μl/well charcoal solution and mix with an 8-channel pipette (4-5 aspirations)

Chill the plate on ice

Centrifuge at 4° C., 2000×g for 15 min

Transfer 100 μl supernatant into an Optiplate, also add 25 μl substrate mixture to 2 empty wells to indicate counting efficiency

Add 200 μl Microscint-40 to all wells and count on a Topcount

Radioimmunoassay

The 11β HSD1 enzyme assay was carried out following the standard operating procedure described above in u-bottom polypropylene 96 well plates or 1.5 ml Eppendorf tubes as indicated for each experiment. Subsequent to stopping the enzyme reaction, 100 μl antibody prepared in buffer 3 unless otherwise indicated was added to test samples and 100 μl buffer 3 was added to the NSB samples. The samples were incubated for 1 hour at 37° C. and the chilled on ice for 15 mins. Dextran coated charcoal (50 μl/sample) prepared to the indicated concentration in buffer 3 was added and the samples were mixed (vortex for tubes and aspiration 5 times with an 8-channel pipette for 96 well plates) and chilled for a further 10 min. The samples were centrifuged at 2000×g for 15 min at 4° C. to pellet the charcoal. Aliquots of the supernatant (100 μl) were transferred to an Optiplate and counted on the Topcount in 150-200 μl Microscint 40. In some experiments, aliquots of supernatant were transferred to scintillation vials and counted on the Tricarb LSC in 5 ml Ultima Gold scintillant.

Inhibition Data

| | | % inhibition of Human 11β HSD1 |
|---|---|---|
| STX No. | Structure | @ 10 μM typical sd ± 5% N=2 |
| 551 | 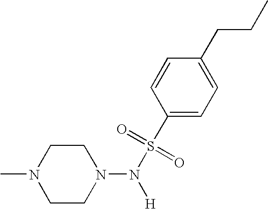 | >35 |
| 578 | 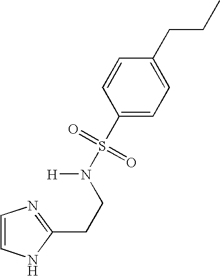 | >35 |
| 586 | 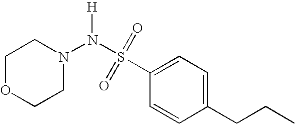 | >35 |

Chemistry Experimental Section and Compound Examples

General. All chemicals were either purchased from the Aldrich Chemical Co. (Gillingham, UK), Lancaster Synthesis (Morecambe, UK) or ACROS Organics (Loughborough, UK). All organic solvents of A.R. grade were supplied by Fisher Scientific (Loughborough, UK).

Thin layer chromatography (TLC) was performed on precoated plates (Merck TLC aluminium sheets silica gel 60 F₂₅₄, Art. No. 5554). Compounds were visualised by either viewing under UV light or treating with an ethanolic solution of phosphomolybdic acid (PMA) followed by heating. Flash chromatography was carried out using Sorbsil C60 silica gel or Isolute® pre-packed Flash Si columns from Argonaut Technologies. Parallel synthesis was performed on either Radleys Carousel reaction stations or Radleys GreenHouse parallel synthesisers. Solvent removal from parallel syntheses was performed on a GeneVac DD4 evaporation system. NMR spectra were recorded with a JEOL GX-270 or Varian-Mercury-400 spectrometer, and chemical shifts are reported in parts per million (ppm, δrelative to tetramethylsilane (TMS) as an internal standard. Mass spectra were recorded at the Mass Spectrometry Service Centre, University of Bath. FAB-MS were carried out using m-nitrobenzyl alcohol (NBA) as the matrix. High performance liquid chromatography (HPLC) analysis was performed with a Waters Delta 600 liquid chromatograph with a Waters 996 photodiode Array Detector using a Waters Radialpack C18, 8×100 mm column. Melting points (Mp) were measured with a Reichert-Jung ThermoGalen Kofler block or a Sanyo Gallenkamp melting point apparatus and are uncorrected.
Synthesis of Benzamides and Arysulphonamides 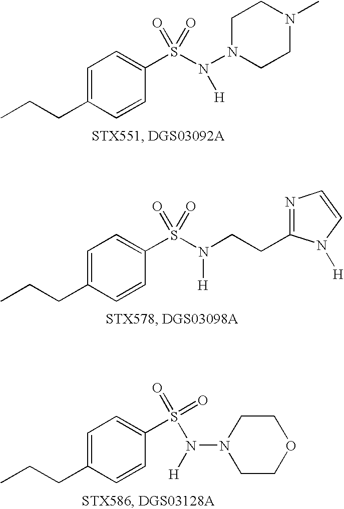
General Method for synthesis of arvisulphonamide (STX551, STX578, STX586)

To a solution of amine (1 eq.) in DMF was added Et₃N (5 eq.), followed by corresponding sulphonyl chloride (1.2 eq.). The reaction mixture was stirred at room temperature under N₂ overnight, poured into water after TLC showed completion of the reaction, and extracted with ethyl acetate, dried (MgSO₄), concentrated under reduced pressure to give the desired sulphonamide as crystalline solid or as a thick syrup. The crude compound was then purified by flash chromatography using EtOAc/hexane (3:2) or CH₂Cl₂/EtOAc (4:1) as eluent to give crystalline solid. Yield 20-80%.

### N-(4-Methylpiperazin-1-yl)-4-propylbenzenesulfonamide (STX551, DGS03092A)

White crystalline solid. mp 153-154° C.; TLC single spot at R_{f} 0.49 (20% ethyl acetate-DCM); HPLC purity 99% (t_{R} 1.9 min in 4% water-methanol); ¹H NMR (400 MHz, DMSO-d6): δ 8.66 (1H, s, NH), 7.71-7.74 (2H, m, ArH), 7.39-7.41 (2H, m, ArH), 2.64 (2H, t, J=7.4 Hz, CH₂), 2.45 (4H, m, 2×CH₂), 2.18 (4H, m, 2×CH₂), 2.06 (3H, s, CH₃), 1.61 (2H, m, CH₂), 0.86 (3H, t, J=7.4 Hz, CH₃); FAB-MS 298 (MH⁺); FAB-HRMS calcd for C₁₄H₂₄N₃O₂S (MH⁺) 298.1589, found 298.1595.

### N-[2-(4H-Imidazol-2-yl)-ethyl]-4-propylbenzenesulfonamide (STX578, DGS03098A)

Pale yellow syrup. TLC single spot at R_{f} 0.51 (20% ethyl acetate-DCM); HPLC purity 96% (t_{R} 2.4 min in 10% water-methanol); ¹H NMR (400 MHz, DMSO-d6): δ 7.63-7.69 (3H, m, ArH and NH), 7.53 (1H, m, ArH), 7.39-7.42 (2H, m, ArH), 7.06 (1H, t, J=1.2 Hz, ArH), 6.84 (1H, t, J=1.2 Hz, ArH), 3.94 (2H, t, J=7.4 Hz, CH₂), 2.65 (2H, t, J=7.4 Hz, CH₂), 1.74-1.81 (2H, m, CH₂), 1.61 (2H, m, CH₂), 0.89 (3H, t, J=7.4 Hz, CH₃); FAB-MS 294 (MH⁺); FAB-HRMS calculated for C₁₄H₁₉N₃O₂S (MH⁺) 294.0361, found 294.0362.

### N-Morpholin-4-yl-4-propyl-benzenesulfonamide (STX586, DGS03128A)

White solid. mp 150-152° C.; TLC single spot at R_{f} 0.46 (20% ethyl acetate-DCM); HPLC purity >99% (t_{R} 1.8 min in 4% water-methanol); ¹H NMR (400 MHz, DMSO-d6): δ 8.79 (1H, s, NH), 7.74-7.77 (2H, m, ArH), 7.39-7.42 (2H, m, ArH), 3.40-3.44 (2H, m, CH₂), 3.30-3.38 (2H, m, CH₂), 2.64 (2H, t, J=7.4 Hz, CH₂), 2.49-2.51 (2H, m, CH₂), 2.44-2.46 (2H, m, CH₂), 1.61 (2H, m, CH₂), 0.86 (3H, t, J=7.4 Hz, CH₃); FAB-MS 285 (MH⁺); FAB-HRMS calculated for C₁₃H₂₁N₂O₃S (MH⁺) 285.1271, found 285.1272.

### DETAILED DESCRIPTION OF FIGURES

Separation of Cortisone and Cortisol

Several solvent systems claiming to separate cortisone from cortisol are detailed in the literature[30, 31]. Before running an assay, 10 mg/ml solutions of cortisone and cortisol were prepared in IMS and 50 μl aliquots were spotted separately onto a silica gel TLC plate 3 cm from the bottom edge and 2.5 cm apart. The plate was run in a TLC tank in 200 ml of CH₂Cl₂:IMS 92:8 v/v [30] until the solvent front reached the top of the plate. The plate was air dried and sprayed with 0.1% Rhodamine B in IMS to visualise the spots. The Table below describes the separation obtained.

TABLE
Separation of cortisone from cortisol by TLC

| | Distance run from origin | Solvent front migration/ |
|---|---|---|
| Steroid | (cm) | steroid migration (cm) |
| Cortisone | 7.5 | 2.3 |
| Cortisol | 4.5 | 3.8 |

The separation was considered adequate for use in an enzyme assay.

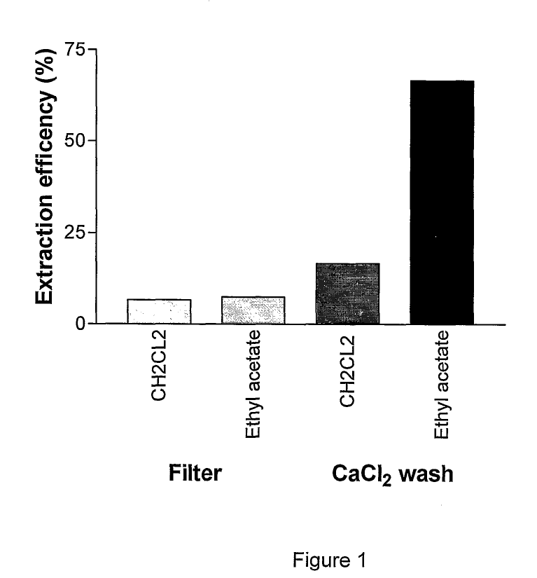

FIG. 1 (Extraction Efficiencies Obtained with Four Extraction Methods)

The literature details several methods of extracting cortisol from aqueous solution[30, 31]. In order to select a method for use, a ¹⁴C-labelled cortisol was obtained from NEN. A stock was prepared in PBS containing 4000 DPM in 50 μl with cold cortisol (1 μg) added as a carrier. The final ethanol concentration was 0.4%. Aliquots of this solution were added to glass tubes (100 μl) and the following extractions were carried out: 1. 1 ml CH₂Cl₂, vortex and pass through phase separating filter paper (Whatman, IPS) 2. 1 ml ethyl acetate, vortex and pass through phase separating filter paper 3. 1 ml CH₂Cl₂ and 200 μl 0.05% CaCl₂, vortex, centrifuge (500×g for 5 min) and remove upper aqueous phase 4. 1 ml ethyl acetate and 200 μl 0.05% CaCl₂, vortex, centrifuge (500×g for 5 min) and collect upper organic phase. The organic phases were dried and the residues were taken up in 100 μl IMS. An aliquot of this (50 μl) was spotted onto a TLC plate and run as before. Following visualisation with Rhodamine B, the spots were scraped into scintillation vials and counted on a liquid scintillation counter (TriCarb) in 5 ml Ultima gold scintillant. Extraction efficiencies were calculated and are given in FIG. 1.

Assay of Human and Rat Hepatic Microsomal 11β-HSD1 Activity using a TLC Separation of Substrate from Product

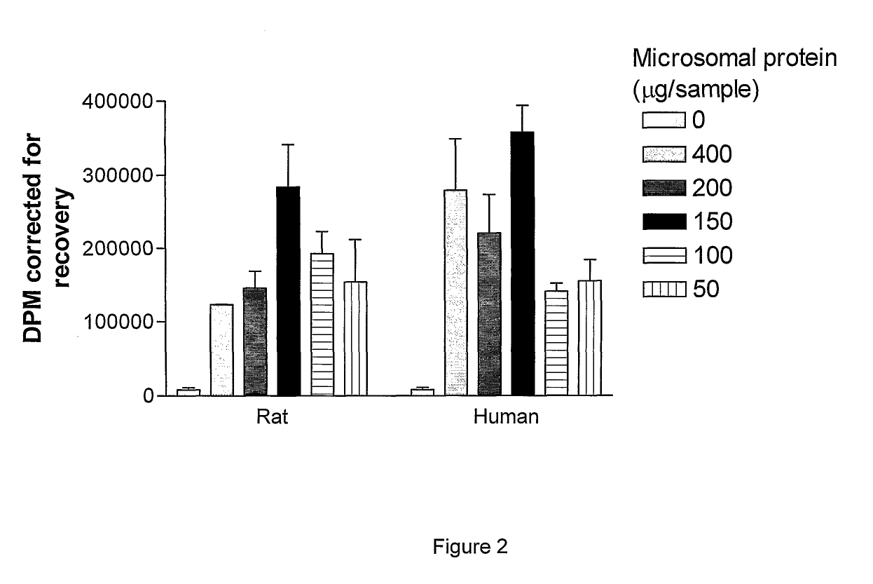

FIG. 2 (Comparison of 11β-HSD1 Activity in Rat and Human Hepatic Microsomes)

This experiment was carried out to compare the enzyme activity in hepatic microsomes from human and rat and to assess minimum microsomal protein concentrations necessary for reasonable measurement of enzyme activity. The assay was carried out in Buffer 2 and the cortisone concentration used was 2 μM containing 0.5 μCi per incubation ³H-cortisone. Rat and human hepatic microsomes were tested at concentrations ranging from 400 μg to 50 μg microsomal protein per incubation in a final incubation volume of 100 μl in glass tubes. Buffer was substituted for microsomal protein for blanks. Samples were incubated for 1 h in a shaking water bath at 37° C. and the assay was stopped by the addition of 1 ml ethyl acetate. To correct for recovery, 50 μl ¹⁴C-cortisol (approximately 4000 DPM per tube) was added to the samples followed by 200 μl 0.05% CaCl₂. The samples were vortexed and centrifuged as detailed in 2.1. The upper organic phase was removed into clean tubes and dried down. The residue was taken up in 100 μl IMS and 50 μl aliquots were spotted onto TLC plates which were run as described in 2.1. DPM were measured on a TriCarb liquid scintillation counter using a dual label programme. Recovery was determined from the DPM obtained in 50 μl ¹⁴C-cortisol solution which was counted with the samples. The results are given in FIG. 2.

It had been expected that there would be higher activity in the rat microsomes but this was not the case. Using 50 μg microsomal protein per well the activities of the rat and human enzymes were quite similar, rat microsomal activity was 0.7 pmol/mg/min and human was 0.5 pmol/mg/min. Good activity was detected in the human microsomes although this was not particularly related to microsomal protein concentration. It was suspected that the concentration range examined was too high so in the next experiment a lower range was tested. Also, the dependence of apparent enzyme activity with incubation time was looked at. A linear relationship between DPM and incubation time would indicate that increases over blank were due to enzyme activity and not due to an artefact.

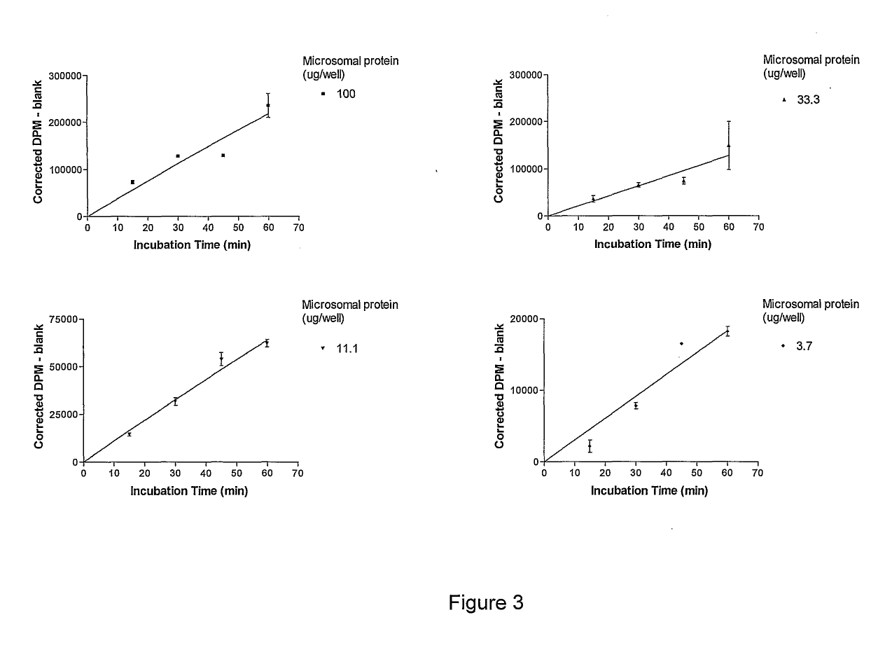

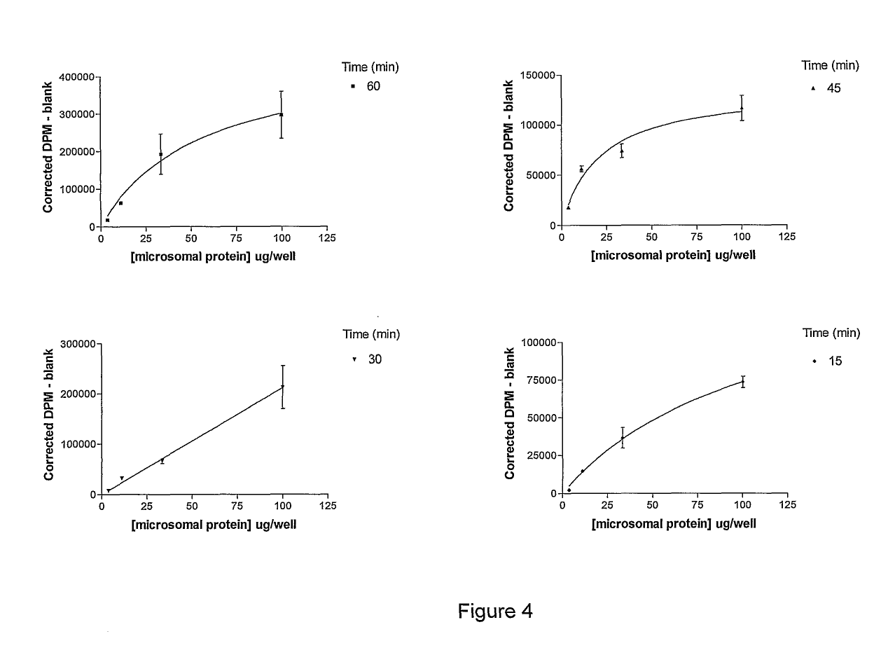

FIG. 3 (Effect of Incubation Time on Human Microsomal 11μ-HSD1 Activity) and FIG. 4 (Effect of Microsomal Protein Concentration on Human Microsomal 11β-HSD1 Activity)

In the next test, the same assay method was followed except that only human hepatic microsomes were examined and the concentration range of these was from 3.7 μg per sample to 100 μg per sample. The samples were incubated for 60 min, 45 min, 30 min or 15 min in a shaking water bath at 37° C. and were stopped, extracted and the substrate and product were separated as detailed above. FIG. 3 and FIG. 4 illustrate the results:

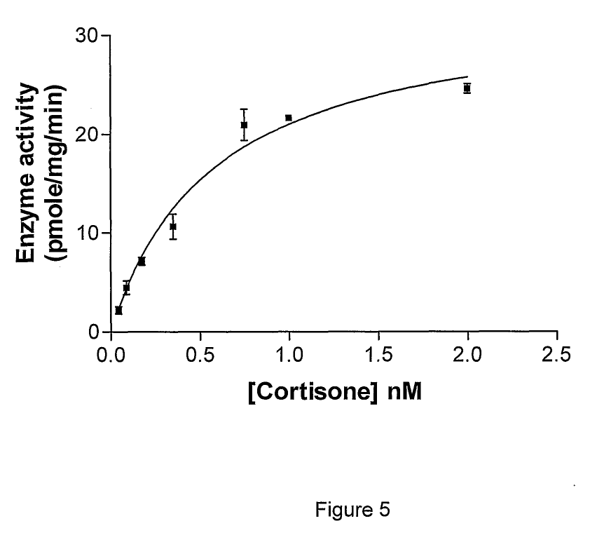

FIG. 5 (Substrate (Cortisone) Saturation Curve for Human Hepatic Microsomal 11βHSD1)

Substrate requirement was examined using the classical assay. The DPM in each group was kept constant (0.5 μCi/sample) and the cold cortisone was varied from 2 μM down to 43.8 nM. The assay was carried out with 10 μg microsomal protein per sample and the incubation time was 30 min at 37° C. The buffer used for this assay was Buffer 1 FIG. 5 shows the data obtained.

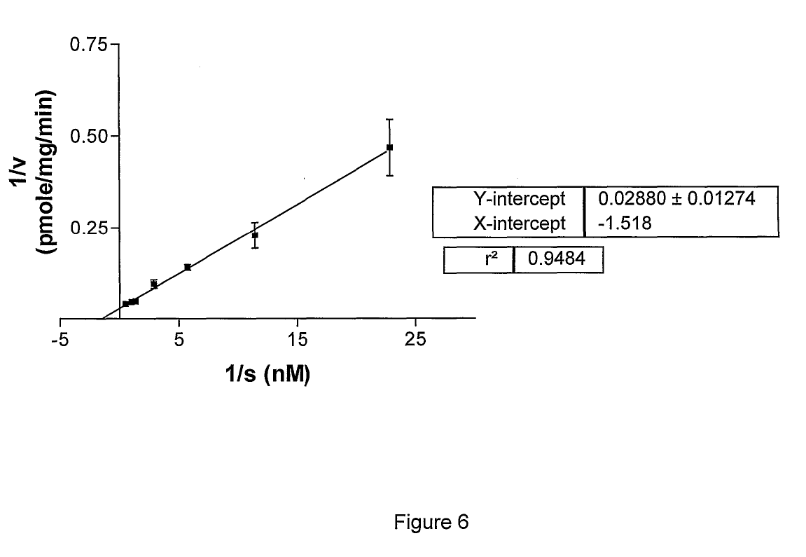

FIG. 6 (Lineweaver-Burke Plot)

A double reciprocal plot of these data (Lineweaver-Burke) gives an apparent Km for cortisone of 660 nM, but it should be noted that it is unlikely that initial enzyme activity rates were measured at the lower [cortisone] over a 30 mins incubation. FIG. 6 shows the Lineweaver-Burke plot obtained:

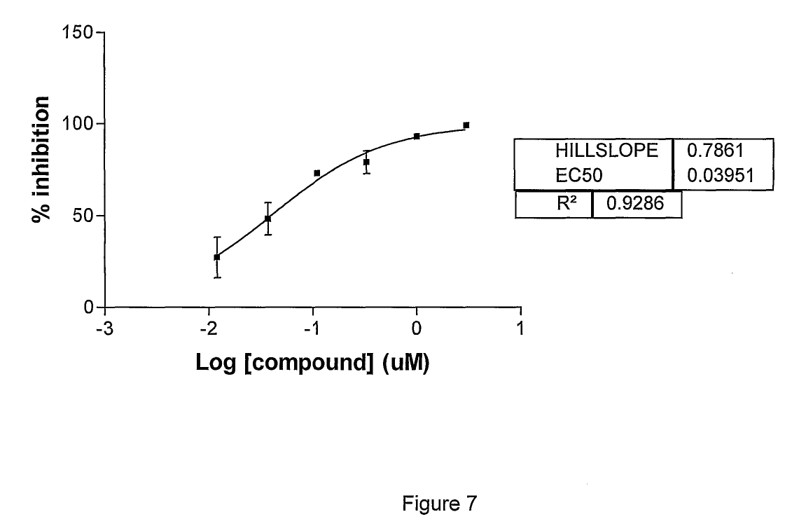

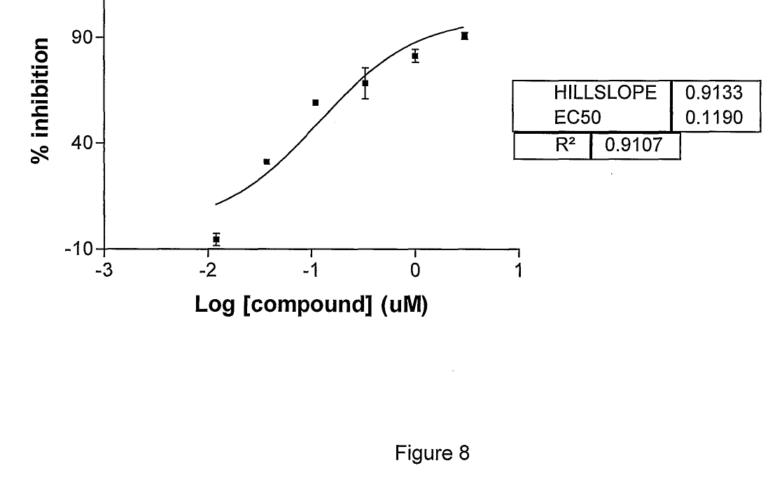

FIG. 7 (IC₅₀ Determination for Glycyrrhetinic Acid) and FIG. 8 (IC₅₀ Determination for Carbenoxolone)

In order to reproduce the inhibition data given in [14], it was decided to use the cortisone concentration quoted in the reference (175 nM) to examine compound activity, even though FIG. 5 and FIG. 6 suggest that this concentration is not saturating with 10 μg microsomal protein per 30 mins incubation at 37° C. The following experiment was carried out with a lower microsomal protein concentration (5 μg) in the same buffer conditions as in the last experiment (Buffer 1) over a 30 mins incubation at 37° C. in the presence of 175 nM cortisone (0.5 μCi/sample). Glycyrrhetinic acid and carbenoxolone were examined at concentrations from 3 μM to 0.012 μM (DMSO concentration 1% throughout) and the data are shown in FIG. 7 and FIG. 8.

The reported IC₅₀ for carbenoxolone is 330 nM [14], which is approximately three times less active than observed in the above experiment. It appears that these assay conditions support good enzyme activity which should be measurable in a 96 well plate method.

Cortisol Immunoassay

An enzyme immunoassay kit was obtained from Assay Designs, Inc. The antibody provided in the kit is a mouse monoclonal reported to cross react 100% with cortisol (the enzyme product) and <0.1% with cortisone (the enzyme substrate). The kit is designed for the analysis of cortisol levels in saliva, urine, serum and plasma and also in tissue culture media, not for microsomal enzyme activity.

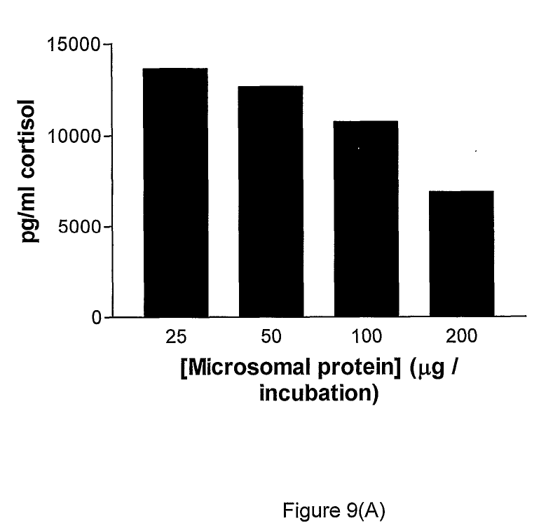

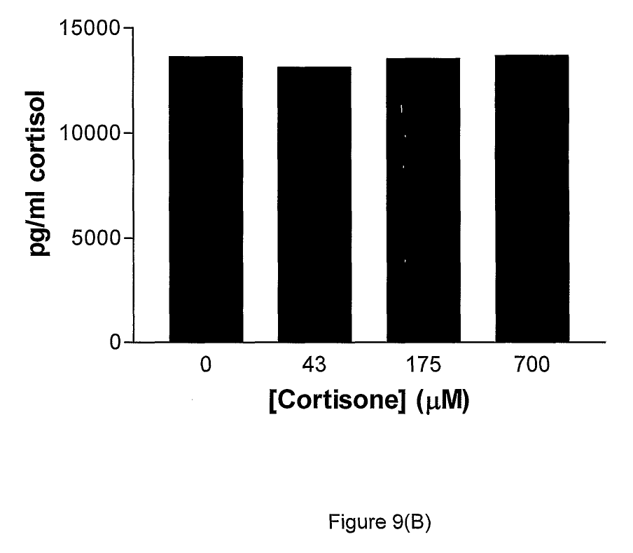

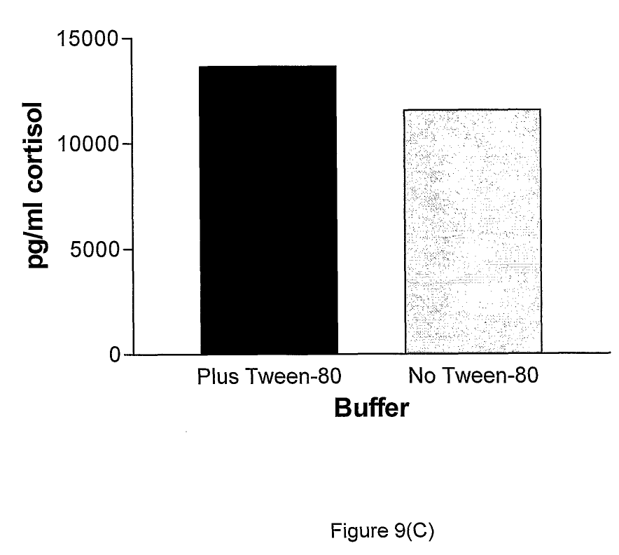

FIG. 9 (11β-HSD1 Activity Measured by Immunoassay)

The methodology for the enzyme assay used with the kit was based on the paper by Barf et al. [14] Human hepatic microsomes were incubated in Buffer 1 at concentrations ranging from 25 μg protein per point to 200 μg protein per point in the presence of cortisone ranging from 44 nM to 700 nM for 1 h. Also, these groups were tested in the presence and absence of 0.9% Tween 80 since this detergent may improve the activity of enzymes involved in steroid metabolism. The basis of the assay is one of competition between the sample cortisol binding and the detector-cortisol binding. The assay detected the cortisol in the standard curve (313 pg/ml to 10,000 pg/ml) as expected but the signal obtained from the enzyme assay samples decreased with increasing microsomal protein concentration, suggesting that the presence of microsomes interfered with the immunoassay. FIGS. 9(A), 9(B) and 9(C) shows some of the data obtained.

FIG. 9(A) shows the effect of protein. Data taken from 700 μM cortisone group tested in the presence of Tween-80 FIG. 9(B) shows the effect of cortisone. Data taken from the 25 μg microsomal protein group tested in the presence of Tween-80 FIG. 9(C) shows the effect of Tween-80. Data taken from the 25 μg microsomal protein group tested in the presence of 700 μM cortisone

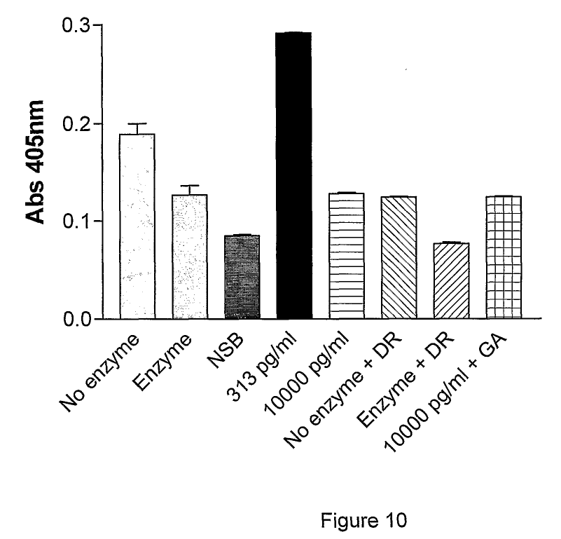

FIG. 10 (Performance of the Cortisol Immunoassay: Various Experimental Designs)

An 11β-HSD1 assay was carried out with 24 μg microsomal protein/sample and 2 μM cortisone substrate in buffer 2 Enzyme activity was also measured in samples following the addition of steroid displacement reagent (kit component) which releases cortisol from cortisol binding protein, if present in the sample. The assay detected the cortisol in the standard curve (313 pg/ml to 10,000 pg/ml). FIG. 10 shows the absorbance at 405 nm obtained for the different groups:

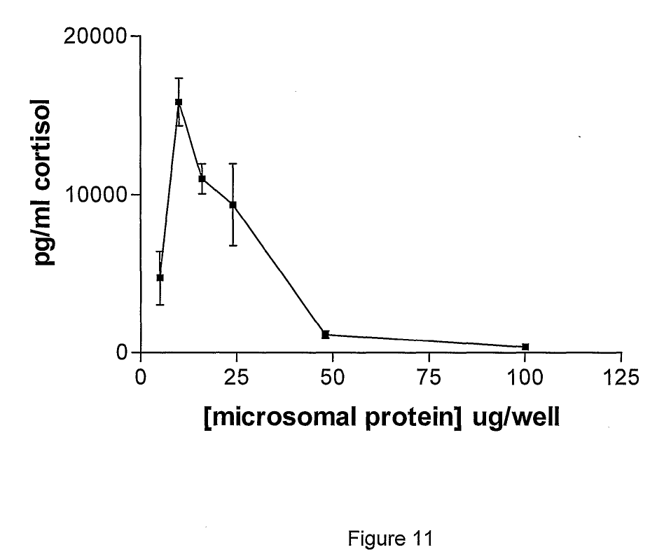

FIG. 11 (Effect of Increasing Microsomal Protein on Measurement of 11β-HSD1 Activity Detected by Assay Designs Immunoassay)

When the enzyme assay data was calculated as enzyme activity using the pg/ml cortisol indicated by the standard curve, the blank value was 47 pg/ml/min incubation and the enzyme activity was 119 pg/ml/min, a signal to noise of 2.5. Although the signal to noise obtained is rather poor, these data demonstrate that the antibody can bind the cortisol:AP conjugate and that this can be displaced by cortisol. An experiment was carried out to examine the effect of slightly increasing the microsomal protein concentration in an attempt to improve the signal to noise obtained. Microsomal protein was tested from 100 μg/incubation down to 5 μg/incubation using 2 μM cortisone in buffer 2. All other conditions were identical to those detailed above. The results are shown in FIG. 11.

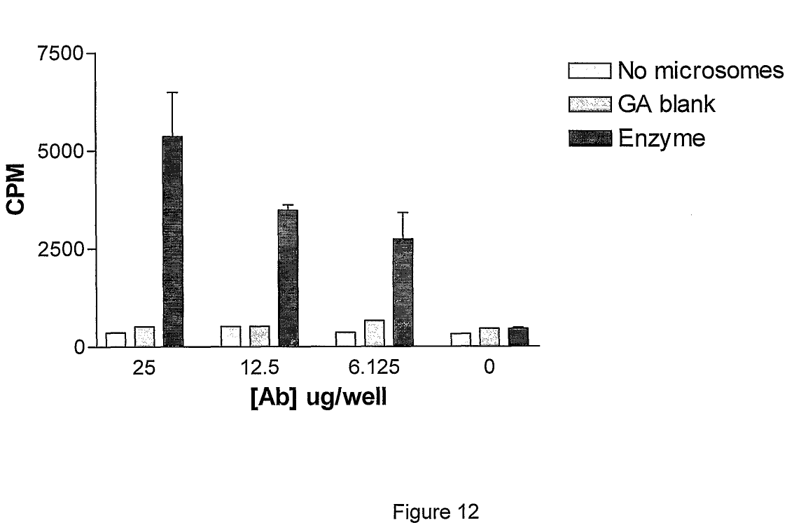

FIG. 12 (Detection of 11β HSD1 Activity by RIA using the Immunotech Anti-Cortisol Antibody)

The next experiment was carried out to assess the Immunotech antibody. The enzyme assay was carried out in Buffer 2. The substrate (cortisone) concentration of 175 nM was chosen from the SPA method described by Barf et al. [14] with 0.5 μCi/well ³H-cortisone. The enzyme assay was carried out in a polypropylene plate in a final incubation volume of 100 μl containing 10 μg/well human hepatic microsomal protein. Blanks had either buffer substituted for microsomal protein or had 10 μl stop solution added prior to the microsomes. The assay was incubated at 37° C. for 30 mins and the reaction was terminated by the addition of the stop solution to all remaining wells. The Immunotech antibody was diluted in Buffer 3 to give 25 μg/100 μl down to 6.25 μg/100 μl. The antibody (100 μl) was added to test wells, 100 μl Buffer 3 was added to the antibody blank wells. The remainder of the procedures followed the 96 well plate RIA protocol exactly. Results demonstrating 11 HSD1 activity using the Immunotech are shown in FIG. 12.

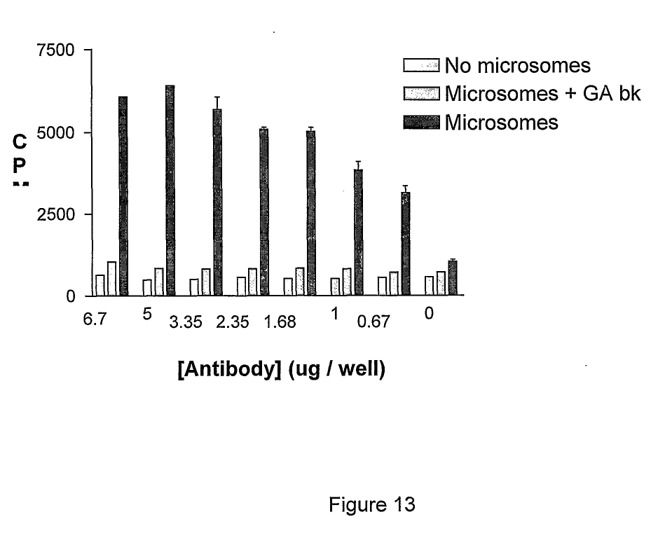

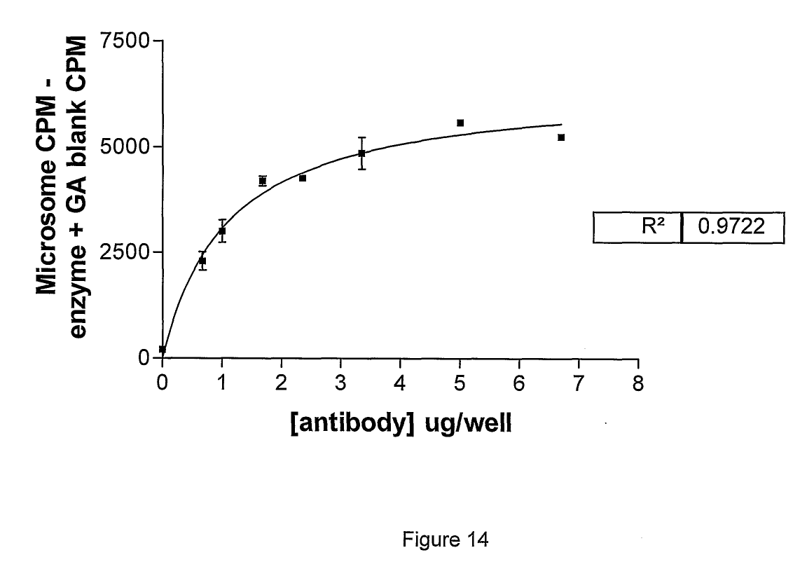

FIG. 13 (Effect of Lowering the Immunotech Antibody Concentration on the Signal to Noise (Microsome Group Compared to GA Blank Group)) and FIG. 14 (Immunotech Antibody Saturation Curve for Detection of 11β HSD1 Activity by RIA)

The antibody titre was examined in the next test, investigating concentrations per well from 6.7 μg down to 0.67 μg. The usual 11β HSD1 assay was carried out except that the microsomal protein concentration was doubled to 20 μg/well in order to get the best signal to noise. The cortisone concentration was 175 nM and the enzyme assay buffer was Buffer 2. Each antibody concentration was tested against a “no enzyme” blank (buffer substituted for microsomes) and a “GA blank” (10 μl stop solution added prior to microsomes) and a control group. The RIA was carried out exactly as indicated in the methods for assay in 96 wells. These results are shown in FIG. 13 and FIG. 14.

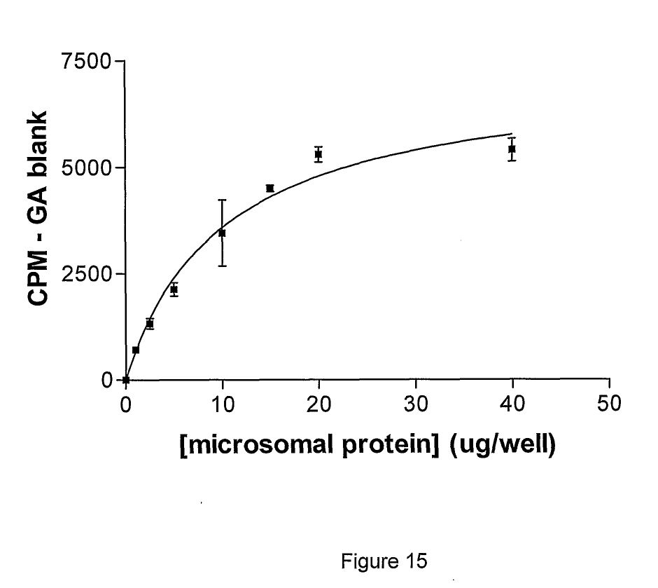

FIG. 15 (Linearity of Human Hepatic Microsomal 11β HSD1 Activity Detected by RIA)

Linearity of enzyme activity with human hepatic microsomal protein concentration using RIA detection was examined in the next test. The usual 11β HSD1 assay was carried out except that the microsomal protein concentration was varied from 40 μg/well down to 1 μg/well. The cortisone concentration was 175 nM and the enzyme assay buffer was Buffer 2. 11β HSD1 activity was linear with protein up to concentrations of 20 μg/well confirming the results obtained with the classical enzyme assay (FIG. 4). Data from these experiments are shown in FIG. 15.

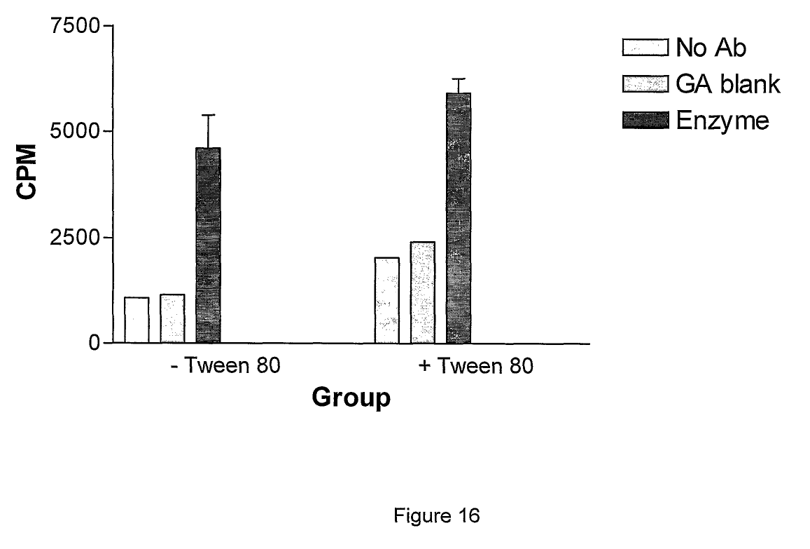

FIG. 16 (Effect of Tween 80 on Detection of Human Hepatic Microsomal 11β HSD1 Activity by RIA)

The effect of including Tween 80 in the enzyme assay buffer was also investigated. This assay was carried out in parallel with the assay above and under the same conditions except that the enzyme assay buffer (Buffer 2) contained 0.05% Tween 80. Microsomal protein was tested at four concentrations. Tween 80 was found to increase the blank CPM, reducing the signal to noise of the assay. The data in FIG. 16 are taken from the group tested with 10 μg/well microsomal protein, but the same effect was seen with all protein concentrations examined.

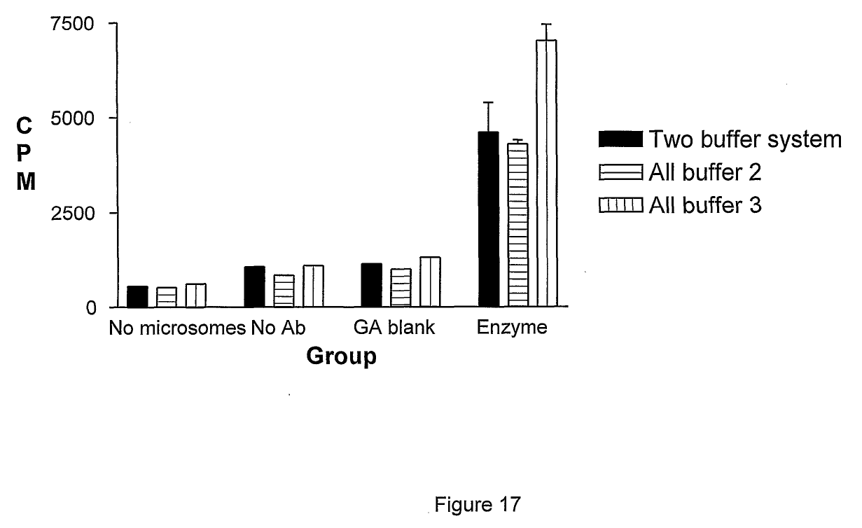

FIG. 17 (Effect of Buffer Systems on Detection of Human Hepatic Microsomal 11βHDS1 Activity by RIA)

To simplify the protocol such that both enzyme assay and RIA stages are carried out in the same buffer, both phases were carried out in either enzyme assay buffer (buffer 2) or buffer 3 (RIA buffer). The microsomal protein concentration used was 10 μg/well and the cortisone concentration was 175 nM. Performing both enzyme assay and RIA in enzyme assay buffer gave similar data to the two buffers system but performing both enzyme assay and RIA in Buffer 3 appeared to improve the data slightly. These results are highlighted in FIG. 17.

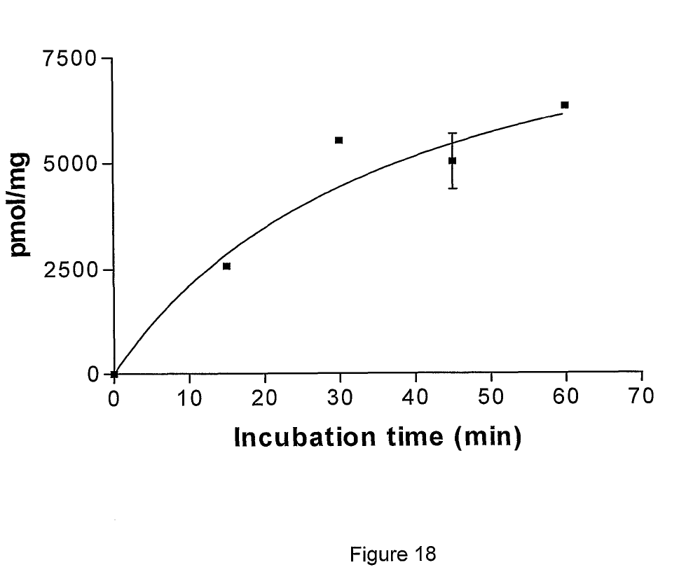

FIG. 18 (Linearity of Human Hepatic Microsomal 11β HSD1 Activity with Incubation Time Detected by RIA)

Linearity of enzyme activity with incubation time was investigated. The enzyme assay was carried out exactly as indicated in the methods section in buffer 3 with 10 μg/well microsomal protein and with 175 nM cortisone. The reaction was stopped at the times indicated in FIG. 18 by the addition of 10 μl stop solution. The RIA was carried out exactly as indicated in the methods section. FIG. 18 illustrates these results.

It is possible that 175 nM substrate is low. The apparent Km observed in the classical 11β HSD1 assay was 660 nM (FIG. 5 and FIG. 6), although these assays are end-point measurement, hence it is not certain that initial rates were measured in the low substrate groups with a 30 min incubation time. However, there are published Km values which suggest that the actual Km for cortisone in human hepatic microsomal 11β HSD1 assays is in the micromolar range [31, 32]. Even though 175 nM substrate is well below the apparent Km, it may not be possible to increase the concentration significantly for two reasons:

(i) If the compounds are competitive with cortisone, the measured inhibition will fall if the substrate is increased above the concentration used in Reference [14].

(ii) Increasing the substrate will reduce the specific activity of the label, reducing the CPM and the sensitivity of the assay—this could be overcome by adding higher concentrations of ³H-cortisone

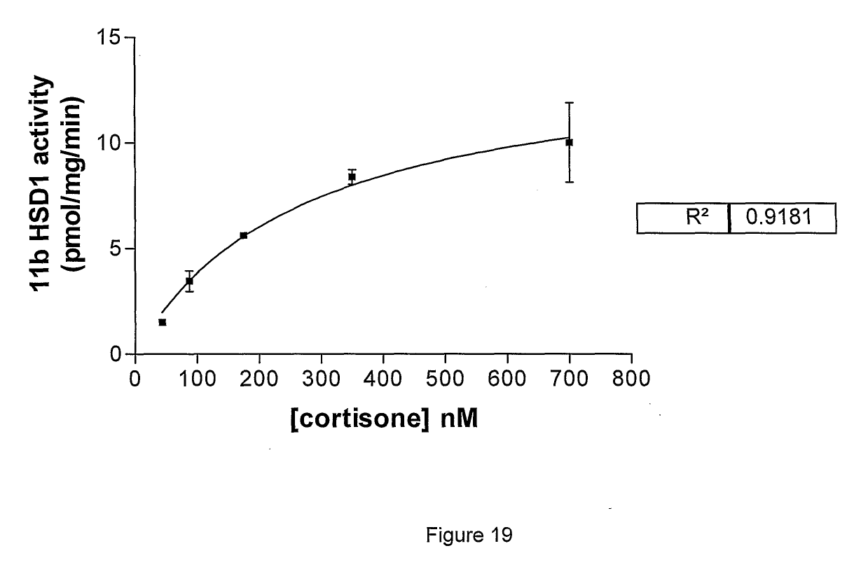

FIG. 19 (Substrate Saturation Curve for Human Hepatic Microsomal 11β HDS1 Activity Detected by RIA)

The substrate saturation effects were examined in the next assay. The enzyme assay was carried out exactly as indicated in the methods section in buffer 3 with 10 μg/well microsomal protein and with [cold cortisone] as indicated. 3H-cortisone was 0.5 μCi/sample throughout. The reaction was stopped after 30 min by the addition of 10 μl stop solution. The RIA was carried out exactly as indicated in the methods section. Results are shown in FIG. 19.

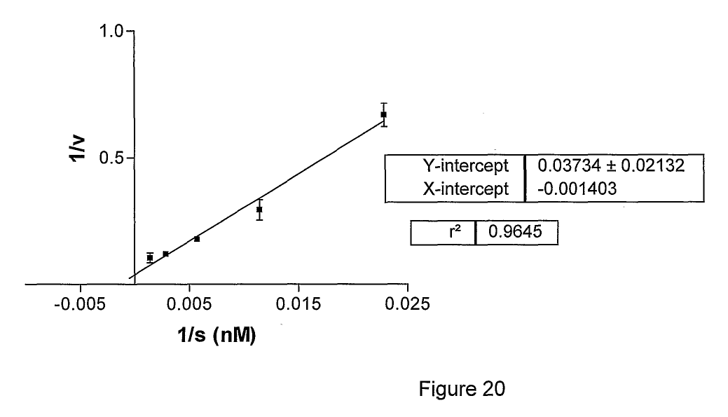

Inspection of the data shown in FIG. 19 shows that 10 μg microsomal protein is not saturated with 175 nM cortisone over an incubation period of 30 mins. The apparent Km (700 nM), determined from the Lineweaver-Burke plot of these data shown in FIG. 20 is very similar to that determined in the classical 11β HSD1 assay (FIG. 6, apparent Km ˜660 nM).

FIG. 20 (Lineweaver-Burke Plot)

Lowering the microsomal protein concentration or the incubation time to fit well within the linear range would partly overcome the problem, but both adjustments would decrease the assay sensitivity. All of the tests carried out so far suggest that even if increasing the microsomal protein from 10 μg/sample to 20 μg/sample does not result in a doubling of enzyme activity, decreasing it from 10 μg/sample to 5 μg/sample does result in a twofold decrease in enzyme activity. Since the purpose of the assay is to monitor inhibitory effects of compounds it is probably a better course of action to leave the assay parameters as they are.

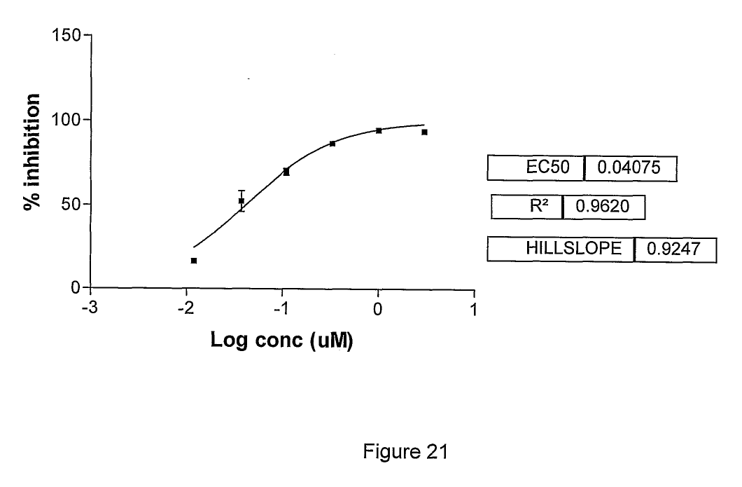

FIG. 21 (IC₅₀ Curve for Inhibition of Human Hepatic Microsomal 11β HSD1 Activity by Glycyrrhetinic Acid)

In order to assess the quality of compound inhibition data obtained in this assay format, an IC₅₀ for Glycyrrhetinic acid was determined in the next test. A 10 mM stock solution of Glycyrrhetinic acid was prepared in 100% DMSO and was further diluted in 100% DMSO to 0.3 mM. This solution was serially diluted in 100% DMSO 1 in 3 to obtain the test range and each solution was diluted in assay buffer (Buffer 3) 1 in 25. These solutions were diluted into the final enzyme reaction 1 in 4 to give assay concentrations from 3 μM down to 0.012 μM in a final [DMSO] of 1%. Controls, NSB (no antibody) and GA blanks (addition of 10 μl stop solution prior to the addition of microsomes) were included with and without the addition of 1% DMSO. Human hepatic microsomal protein was tested at 10 μg/well and the substrate concentration (cortisone) was 175 nM, 0.5 μCi/well. All other procedures were as indicated in the methods section. GA inhibition data are shown in FIG. 21.

The assay control and blank CPM are given in the Table below.

TABLE
Control and blank CPM obtained in the Glycyrrhetinic
acid IC₅₀ assay showing effect of 1% DMSO and
signal to noise ratio obtained.

| Group | 1% DMSO | No DMSO |
|---|---|---|
| NSB | 670 | 661 |
| GA blank | 640 | 660 |
| Control | 3515 | 2583 |
| Signal to noise | 5 fold | 4 fold |

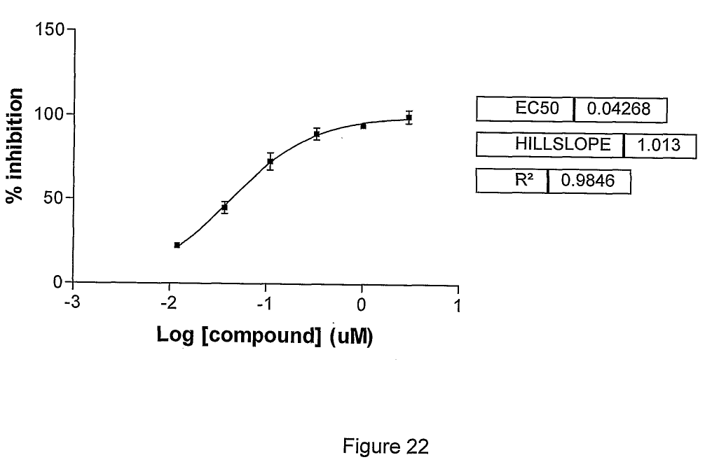

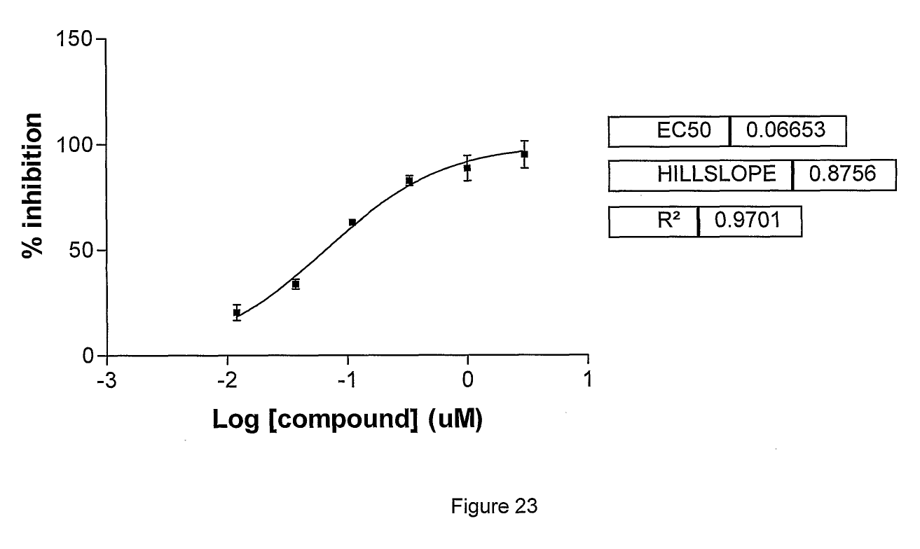

FIG. 22 (IC₅₀ Curve for Inhibition of Human Hepatic Microsomal 11β HSD1 Activity by Glycyrrhetinic Acid in the Presence of 350 nM Cortisone) and FIG. 23 (IC₅₀ Curve for Inhibition of Human Hepatic Microsomal 11β HSD1 Activity by Carbenoxolone in the Presence of 350 nM Cortisone)

In the next experiment, Glycyrrhetinic acid and its hemisuccinate ester, carbenoxolone, were tested for IC₅₀ determination. In view of the higher than expected inhibitory activity obtained with Glycyrrhetinic acid (FIG. 22) in the last test and the apparent non-saturation obtained with 175 nM cortisone (FIG. 19), the substrate concentration was increased to 350 nM (0.5 μCi/well). In addition, the final DMSO concentration was reduced to 0.3%. The assay was performed as indicated in the Methods in RIA buffer. Results with some of these compounds are shown in FIG. 22 and FIG. 23.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

### REFERENCES

1. Hammond, G H (1990): Molecular properties of corticosteroid binding globulin and sex-steroid binding proteins. Endocr. Rev. 11, 65-79.
2. Gomez-Sanchez E P, Gomex-Sanchez C E (1997): First there was one, then two . . . why not more 11 β-Hydroxysteroid Dehydrogenases? Endocrinology vol. 138, 12.
3. Krozowski Z S, Funder J W (1983): Renal mineralocorticosterone receptors and hippocampal corticosterone binding species have identical intrinsic steroid specificity. Proc. Natl. Sci. USA 80: 6056-60
4. Ulick S, Levine L S, Gunczler P, Zanconato G, Ramirez L C, Rauh W, Rosier A, Bradlow H L, Mew M I (1979): A syndrome of apparent mineralocorticoid excess associated with defects in the peripheral metabolism of cortisol. J. Clin. Endo. And Metab. 49: 757-64.
5. Edwards C R W, Stewart P M, Burt D, Brett L, McIntyre M A, Sutanto W S, Kloet E R, Monder C (1998): Localisation of 11β-HSD-tissue specific protector of the mineralocorticoid receptor. Lancet 2: 986-989.
6. Moore C C D, Melloh S H, Murai I, Siiteri P K, Miller W L (1993): Structure and function of the hepatic form of 11β-HSD in the squirrel monkey, an animal model of glucocorticoid resistance. Endocrinology 133: 368-375.
7. Kotelevtsev Y V, Iamieson P M, Best R, Stewart F, Edwards C R W, Seckl J R, Mullins I I (1996): Inactivation of 11 β-HSD type 1 by gene targeting in mice. Endocrinology Res. 22: 791-792.
8. Ricketts M L, Verhaeg J M, Bujalska I, Howie A J, Rainey W E, Stewart P M (1998): Immunohistochemical localisation of type 1 11 β-HSD in human tissues. I. Clin. Endoc. Metab. 83: 1325-35.
9. Stewart P M, Sheppard M C (1992): Novel aspects of hormone action: intracellular ligand supply and its control by a series of tissue specific enzymes. Molecular and Cellular Endocrinology 83: C13-C18.
10. Seckl J R, Chapman K E (1997): The 11 β-HSD system, a determinant of glucocorticoid and mineralocorticoid action. Medical and physiological aspects. European I. Biochem. 249: 361-364.
11. Maser E (1998): 11 β-HSD responsible for carbonyl reduction of the tobacco-specific nitrosoamine in mouse lung microsomes. Cancer Res. 58: 2996-3003.
12. Walker B R, Stewart P M, Shackleton C H L, Padfield P L, Edwards C R W (1993): Deficient inactivation of cortisol by 11 β-HSD in essential hypertension. Clin. Endocr. 38: 221-227.
13. Daynes R A, Araneo B A (1998): Contrasting effects of glucocorticoids on the capacity of T-cells to produce the growth factors interleukin-2 and interleukin-4. Eur. J. Immunol. 19: 2319-2324.
14. Barf, T. et al., (2002), Arylsulfonamidothiazoles as a new class of potential antidiabetic drugs. Discovery of potent and selective inhibitors of the 11β-Hydroxysteroid Dehydrogenase Type 1. J. Med. Chem., 45, 3813-3815.
15. Matassa, Victor G. et. al. J. Med. Chem.; 33(9); 1990; 2621.
16. This compound is synthesised in the literature and the NMR spectrum is reported, however the spectrum obtained here differs from that in the literature. Baraldi, Pier Giovanni et. al.; Bioorg. & Med. Chem. Lett.; 10; 2002, 1611.
17. Horaguchi, Takaaki; Matsuda, Shinichi; Tanemura, Kiyoshi; Suzuki, Tsuneo. J. Heterocyclic Chem.; 24; 1987; 965.
18. Plé, Patrick A., Marnett, Lawrence J.; J. Heterocyclic Chem.; 25; 1988; 1271.
19. Rao, U. and Balasubramanian, K.K.; Tetrahedron Lett.; 24; 1983; 5023.
20. Bordwell, F. G. and Stange, Hugo; J. Amer. Chem. Soc.; 77; 1955; 5939.
21. Elderfield, Robert C.; Williamson, Thurmond A.; Gensler, Walter J.; Kremer, Chester B.; J. Org. Chem.; 12; 1947; 405.
22. For 6-nitro-2,3-dimethylquinoxaline see: Barluenga, Jose; Aznar, Fernando; Liz, Ramon; Cabal, Maria-Paz; Synthesis; 3; 1985; 313, then for 6-amino-2,3-dimethylquinoxaline: Salon, Jozef; Milata, Viktor; Pronayova, Nadezda; Lesko, Jan; Collect Czech. Chem. Commun.; 66; 11; 2001; 1691.
23. Klicnar, J.; and Kosek, F.; Collect. Czech. Chem. Commun.; 30; 1965; 3102.
24. Gloster, Daniel F.; Cincotta, Louis; Foley, James W.; J. Heterocyclic Chem.; 36; 1999; 25.
25. The same reduction was carried out using SnCl₂ by: Case et al.; J. Amer. Chem. Soc.; 81; 1959; 6297.
26. Modified procedure from similar compound described in U.S. Pat. No. 6,355,796 (Example 20)
27. Hollfelder, F.; Kirby, A. J.; Tawfik, D. S.; Kikuchi, K.; Hilvert, D.; J. Amer. Chem. Soc.; 122 (6); 2000; 1022-1029
28. Fujimoto, M.; Okabe, K.; Chem. Pharm. Bull.; 10; 1962; 572-575.
29. Kawamura, T.; Yagi, N.; Sugawara, H.; Yamahata, K.; Takada, M.; Chem. Pharm. Bull.; 28; 1; 1980; 268-276.
30. Stewart, P. M. and Mason, J. I., (1995), Cortisol to cortisone: Glucocorticoid to mineralocortcoid. Steriods, 60, 143-146.
31. Escher, G. et al., (1995), Furosemide inhibits 11β-Hydroxysteroid Dehydrogenase in vitro and in vivo. Endocrinology, 136, 1759-1765.
32. Hult, M. et. al., (1998), Selective inhibition of human type 1 11β-hydroxysteroid dehydrogenase by synthetic steroids and xenobiotics. FEBS Letters, 441, 25-28.
33. Diederich S, Grossmann C, Hanke B, Quinkler M, Herrmann M, Bahr V, Oelkers W (2000): In the search for specific inhibitors of human 11 β-HSD: chenodeoxycholic acid selectively inhibits 11 β-HSD type 1. Europ. J. Endocrin. 142: 200-207.

1. A compound having Formula I
R₁—SO₂NR₃-L-R₂ Formula I
wherein R₁ is an optionally substituted phenyl ring;
R₂ is a heterocyclic ring;
R₃ is H or a hydrocarbyl group; and
L is an optional acyclic linker;
wherein when R₂ is a five-membered aromatic heterocyclic ring, L is present.

2. A compound according to claim 1 wherein R₁ is substituted.

3. A compound according to claim 1 or 2 wherein R₁ is substituted with one or more hydrocarbon groups.

4. A compound according to claim 1, 2 or 3 wherein R₁ is substituted with one or more alkyl groups.

5. A compound according to any one of the preceding claims wherein R₁ is substituted with one or more C₁₋₅ alkyl groups.

6. A compound according to any one of the preceding claims wherein R₁ is substituted at the para position of the phenyl ring.

7. A compound according to any one of the preceding claims wherein R₁ is

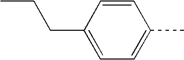

8. A compound according to any one of the preceding claims wherein R₂ is a five or six membered ring.

9. A compound according to any one of the preceding claims wherein R₂ comprises carbon and a hetero atom selected from O and N.

10. A compound according to any one of the preceding claims wherein R₂ comprises carbon and two hetero atoms selected from O and N.

11. A compound according to any one of the preceding claims wherein R₂ has the formula 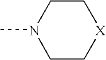
wherein X is selected from the group consisting of S, O and NR₄ wherein R₄ is H or a hydrocarbyl group.

12. A compound according to any one of the preceding claims wherein R₂ is selected from the group consisting of:

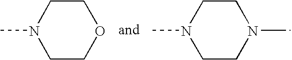

13. A compound according to any one of claims 1 to 10 wherein R₂ is

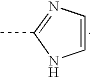

14. A compound according to any one of the preceding claims wherein R₃ is H or a hydrocarbon group.

15. A compound according to any one of the preceding claims wherein R₃ is H or an alkyl group.

16. A compound according to any one of the preceding claims wherein R₃ is H or a C₁₋₅ alkyl group.

17. A compound according to any one of the preceding claims wherein R₃ is H.

18. A compound according to any one of the preceding claims wherein L is present.

19. A compound according to any one of the preceding claims wherein L is a divalent hydrocarbyl linker group.

20. A compound according to any one of the preceding claims wherein L has the formula C_{y}H_{2y} wherein y is an integer from 1 to 10.

21. A compound according to any one of the preceding claims wherein L has the formula (CH₂)ₙ wherein n is an integer from 1 to 10.

22. A compound according to claim 21 wherein n is an integer from 1 to 5.

23. A compound according to any one of claims 19 to 22 wherein L is —CH₂CH₂—.

24. A compound according to any one of the preceding claims selected from

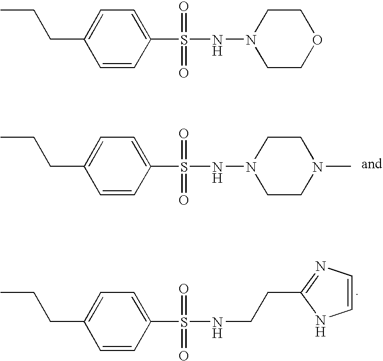

25. A pharmaceutical composition comprising a compound according to any one of claims 1 to 24 optionally admixed with a pharmaceutically acceptable carrier, diluent, excipient or adjuvant.

26. A compound according to any one of claims 1 to 24 for use in medicine.

27. Use of a compound according to any one of claims 1 to 24 in the manufacture of a medicament for use in the therapy of a condition or disease associated with 11β-HSD.

28. Use according to claim 27 wherein the condition or disease is selected from the group consisting of metabolic disorders such as diabetes and obesity; cardiovascular disorders such as hypertension; glaucoma; inflammatory disorders such as arthritis or asthma; immune disorders; bone disorders such as osteoporosis; cancer; intrauterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; and endometrial cancer.

29. Use of a compound according to any one of claims 1 to 24 in the manufacture of a medicament for use in the therapy of a condition or disease associated with adverse 11β-HSD levels.

30. Use of a compound according to any one of claims 1 to 24 in the manufacture of a pharmaceutical for modulating 11β-HSD activity.

31. Use of a compound according to any one of claims 1 to 24 in the manufacture of a pharmaceutical for inhibiting 11β-HSD activity.

32. The invention of any one of claims 27 to 31 wherein 11β-HSD is 11β-HSD Type 1.

33. The invention of any one of claims 27 to 31 wherein 11β-HSD is 11β-HSD Type 2.

34. A compound as substantially hereinbefore described with reference to any one of the Examples.

35. A composition as substantially hereinbefore described with reference to any one of the Examples.

36. A method as substantially hereinbefore described with reference to any one of the Examples.

37. A use as substantially hereinbefore described with reference to any one of the Examples.
